(19) United States
(12) Patent Application Publication
(10) Pub. No.: US 2024/0374711 A1
(43) Pub. Date: Nov. 14, 2024

(54) RECOMBINANT NEWCASTLE DISEASE VIRUS (rNDV) VECTORS AND METHODS OF USING THE SAME

(71) Applicants: Auburn University, Auburn, AL (US); The United States of America, as represented by the Secretary of Agriculture, Beltsville, MD (US)

(72) Inventors: Haroldo E. TORO GUZMAN, Auburn, AL (US); Qingzhong YU, Beltsville, MD (US)

(21) Appl. No.: 18/569,134

(22) PCT Filed: Jun. 13, 2022
(86) PCT No.: PCT/US2022/033282

§ 371 Date: Dec. 11, 2023

Related U.S. Application Data
(60) Provisional application No. 63/209,632, filed on Jun. 11, 2021.

(51) Int. Cl. A61K 39/215; A61K 39/00; A61P 37/04; C12N 15/86

(52) U.S. Cl. CPC A61K 39/215; A61P 37/04; C12N 15/86; A61K 2039/5254; A61K 2039/5256; A61K 2039/55522; C12N 2740/13022; C12N 2740/13034; C12N 2760/18143

(57) ABSTRACT

Disclosed are recombinant Newcastle disease virus (rNDV) vectors, related polynucleotides, and methods for eliciting an immune response against avian infectious bronchitis virus (IBV) or vaccinating against IBV.

### CROSS-REFERENCE TO RELATED APPLICATIONS

This application claims the benefit under 35 U.S.C. § 119 (e) to U.S. Provisional Application No. 63/209,632, filed on Jun. 11, 2021, the content of which is incorporated herein by reference in its entirety.

### STATEMENT REGARDING FEDERALLY SPONSORED RESEARCH OR DEVELOPMENT

This invention was made with government support under 2015-68004-23131 awarded by the U.S. Department of Agriculture (USDA), National Institute of Food and Agriculture (NIFA). The government has certain rights in the invention

### SEQUENCE LISTING

A Sequence Listing accompanies this application and is submitted as an ASCII text file of the sequence listing named “169996_00475_ST25.txt” which is 372,509 bytes in size and was created on Jun. 13, 2022. The sequence listing is electronically submitted via EFS-Web with the application and is incorporated herein by reference in its entirety.

### BACKGROUND

The present invention relates generally to the field of recombinant viral vectors, polynucleotides, and methods for immunizing against coronaviruses. In particular, the invention relates to recombinant Newcastle disease virus vectors expressing infectious bronchitis virus (IBV) spike ectodomain proteins and granulocyte-macrophage colony-stimulating factor (GM-CSF), kits comprising the same, and methods for immunizing avian species against infection by IBV using the aforementioned vectors.

Infectious bronchitis virus (IBV) remains a major cause for economic losses in the poultry industry. IBV's evolutionary success relies on genetically diverse populations, which allow for quick adaptation to changes in selective pressure (1). The increasing number of Arkansas (Ark) vaccine-like viruses isolated from outbreaks of disease in chickens vaccinated with attenuated Ark, shows that live vaccine viruses augment the likelihood of recombination events and subsequently increases IBV's diversity and the virus' fitness in the environment (2, 3, 4, 5). In addition to virus populations originating directly from vaccine viruses, accumulating evidence shows that subpopulations also emerge from wild Ark viruses as result of immune selection in vaccinated chickens (6, 7, 8). Use of recombinant IBV type-specific vaccines instead of varying serotype live vaccines in the poultry industry should reduce emergence of novel IBV.

Unfortunately, expression of the IBV spike(S) S1 or S2 subunits from different viral vectors has shown varying protection levels against IBV challenge (9, 10, 11). Somewhat better results have been obtained using the whole S (12, 13) or the S ectodomain (Se), i.e. S1 extended by the S2 ectodomain (14). Expressing only the Se, which cannot be inserted into the viral envelope in a recombinant viral vector, has the advantage over expression of the complete S protein, in that it avoids the concern that the expression of the IBV S protein on the surface of the virus vector could have the potential to extend its tropism. Therefore, there is a need in the art for improved IBV vaccines.

### SUMMARY

Disclosed are recombinant spike ectodomain proteins, compositions, vectors, kits, and methods for inducing an immune response against avian infectious bronchitis virus (IBV). In particular, the recombinant proteins, compositions, vectors, kits, and methods may be utilized to immunize poultry against disease associated with IBV infection or to protect poultry from IBV infection.

In one aspect of the current disclosure, recombinant Newcastle disease virus vectors are provided. In some embodiments, the vectors comprise: a nucleic acid encoding an infectious bronchitis virus (IBV) spike ectodomain (Se); and a nucleic acid encoding granulocyte-macrophage colony-stimulating factor (GM-CSF). In some embodiments, the amino acid sequence of GM-CSF has at least 90% sequence similarity to SEQ ID NO: 121. In some embodiments, the Se is derived from Arkansas strain IBV. In some embodiments, the Se comprises a multimerization domain. In some embodiments, the multimerization domain is a heterologous multimerization domain. In some embodiments, the amino acid sequence of the multimerization domain is SEQ ID NO: 122. In some embodiments, the recombinant Newcastle disease virus is LaSota strain of Newcastle disease virus. In some embodiments, the IBV spike ectodomain has the amino acid sequence SEQ ID NO: 120.

In another aspect of the current disclosure, polynucleotides are provided. In some embodiments, the polynucleotides comprise: (i) a nucleic acid encoding recombinant Newcastle disease virus; (ii) a nucleic acid encoding infectious bronchitis virus (IBV) spike ectodomain (Se); and (iii) a nucleic acid encoding granulocyte-macrophage colony-stimulating factor (GM-CSF); wherein (i)-(iii) are operably linked to one or more promoters. In some embodiments, the amino acid sequence of GM-CSF has at least 90% sequence similarity to SEQ ID NO: 121. In some embodiments, the Se is derived from Arkansas strain IBV. In some embodiments, the Se comprises a multimerization domain. In some embodiments, the multimerization domain is a heterologous multimerization domain. In some embodiments, the multimerization domain is SEQ ID NO: 122. In some embodiments, the recombinant Newcastle disease virus is LaSota strain of Newcastle disease virus. In some embodiments, the amino acid sequence of the Se is SEQ ID NO: 120. In some embodiments, the polynucleotides have the nucleotide sequence SEQ ID NO: 124.

In another aspect of the current disclosure, pharmaceutical compositions are provided. In some embodiments, the pharmaceutical compositions comprise a recombinant Newcastle disease virus vector comprising: a nucleic acid encoding an infectious bronchitis virus (IBV) spike ectodomain (Se); and a nucleic acid encoding granulocyte-macrophage colony-stimulating factor (GM-CSF); and a pharmaceutically acceptable carrier. In some embodiments, the amino acid sequence of GM-CSF has at least 90% sequence similarity to SEQ ID NO: 121. In some embodiments, the Se is derived from Arkansas strain IBV. In some embodiments, the Se comprises a multimerization domain. In some embodiments, the multimerization domain is a heterologous multimerization domain. In some embodiments, the amino acid sequence of the multimerization domain is SEQ ID NO: 122. In some embodiments, the recombinant Newcastle disease virus is LaSota strain of Newcastle disease virus. In some embodiments, the IBV spike ectodomain has the amino acid sequence SEQ ID NO: 120.

In another aspect of the current disclosure, methods of eliciting an immune response against infectious bronchitis virus (IBV) are provided. In some embodiments, the methods comprise: administering an effective amount of a pharmaceutical composition comprising a recombinant Newcastle disease virus vector comprising: a nucleic acid encoding an infectious bronchitis virus (IBV) spike ectodomain (Se); and a nucleic acid encoding granulocyte-macrophage colony-stimulating factor (GM-CSF); and a pharmaceutically acceptable carrier; to a subject to elicit an immune response against IBV. In some embodiments, the amino acid sequence of GM-CSF has at least 90% sequence similarity to SEQ ID NO: 121. In some embodiments, the Se is derived from Arkansas strain IBV. In some embodiments, the Se comprises a multimerization domain. In some embodiments, the multimerization domain is a heterologous multimerization domain. In some embodiments, the amino acid sequence of the multimerization domain is SEQ ID NO: 122. In some embodiments, the recombinant Newcastle disease virus is LaSota strain of Newcastle disease virus. In some embodiments, the IBV spike ectodomain has the amino acid sequence SEQ ID NO: 120. In some embodiments, the methods further comprise administering a live-attenuated IBV vaccine to the subject. In some embodiments, the live-attenuated IBV vaccine is a Mass strain live-attenuated vaccine. In some embodiments, the subject is a chicken. In some embodiments, subjects administered the pharmaceutical composition exhibit greater protection against challenge by virulent IBV relative to subjects administered a composition not comprising a polynucleotide encoding GM-CSF.

In another aspect of the current disclosure, methods of vaccinating a subject against infectious bronchitis virus (IBV) are provided. In some embodiments, the methods comprise: administering an effective amount of a pharmaceutical composition comprising a recombinant Newcastle disease virus vector comprising: a nucleic acid encoding an infectious bronchitis virus (IBV) spike ectodomain (Se); and a nucleic acid encoding granulocyte-macrophage colony-stimulating factor (GM-CSF); and a pharmaceutically acceptable carrier; to a subject to vaccinate the subject against IBV. In some embodiments, the amino acid sequence of GM-CSF has at least 90% sequence similarity to SEQ ID NO: 121. In some embodiments, the Se is derived from Arkansas strain IBV. In some embodiments, the Se comprises a multimerization domain. In some embodiments, the multimerization domain is a heterologous multimerization domain. In some embodiments, the amino acid sequence of the multimerization domain is SEQ ID NO: 122. In some embodiments, the recombinant Newcastle disease virus is LaSota strain of Newcastle disease virus. In some embodiments, the IBV spike ectodomain has the amino acid sequence SEQ ID NO: 120. In some embodiments, the methods further comprise administering a live-attenuated IBV vaccine to the subject. In some embodiments, the live-attenuated IBV vaccine is a Mass strain live-attenuated vaccine. In some embodiments, the subject is a chicken. In some embodiments, subjects administered the pharmaceutical composition exhibit greater protection against challenge by virulent IBV relative to subjects administered a composition not comprising a polynucleotide encoding GM-CSF.

In another aspect of the current disclosure, methods of generating recombinant Newcastle disease virus vectors are provided. In some embodiments, the methods comprise: expressing a polynucleotide comprising: (i) a nucleic acid encoding recombinant Newcastle disease virus; (ii) a nucleic acid encoding infectious bronchitis virus (IBV) spike ectodomain (Se); and (iii) a nucleic acid encoding granulocyte-macrophage colony-stimulating factor (GM-CSF); wherein (i)-(iii) are operably linked to one or more promoters in a cell to generate a recombinant Newcastle disease virus vector. In some embodiments, the amino acid sequence of GM-CSF has at least 90% sequence similarity to SEQ ID NO: 121. In some embodiments, the Se is derived from Arkansas strain IBV. In some embodiments, the Se comprises a multimerization domain. In some embodiments, the multimerization domain is a heterologous multimerization domain. In some embodiments, the multimerization domain is SEQ ID NO: 3. In some embodiments, the recombinant Newcastle disease virus is LaSota strain of Newcastle disease virus. In some embodiments, the amino acid sequence of the Se is SEQ ID NO: 120.

### BRIEF DESCRIPTION OF THE DRAWINGS

FIG. 1. Expression of IBV Se and NDV HN proteins by IFA. DF-1 cells were infected at 0.01 MOI with LS and rLS/ArkSe.GMCSF, respectively. At 24 hrs post-infection, cells were fixed and stained with a mixture of chicken anti-Ark-type IBV serum and mouse anti-NDV HN Mab, followed by a mixture of FITC-labeled goat anti-chicken IgG and Alexa Fluor® 568 labeled goat anti-mouse IgG. Finally, the infected cells were stained with DAPI. Green, red and blue fluorescent images taken in the same field of virus-infected cells were merged into a single image. Bars represent 100 μm in length.

FIG. 2. NDV antibodies determined by hemagglutination inhibition (HI) assay using chicken sera (n=19/group) collected 23 days after single vaccination with rLS/ArkSe or rLS/ArkSe.GMCSF. Data analyzed by ANOVA with Tukey's multiple comparison post-test. Different letters indicate significant differences at P<0.05.

FIG. 3A-B. Protection induced by single vaccination in chickens (n=19-20/group) at 7 days of age with 10⁶ EID₅₀/bird of rLS/ArkSe or rLS/ArkSe.GMCSF. Chickens were challenged with 10⁴ EID₅₀/bird of virulent Ark 24 days post-vaccination and respiratory signs and viral load (relative IBV RNA) determined 5 days after challenge. UV/UC=unvaccinated/unchallenged; UV/C=UC/challenged controls. (A) Respiratory sign scores for each chicken are shown and means are indicated by horizontal lines. Data analyzed by Kruskal-Wallis test and Dunn posttest. (B) Relative IBV RNA determined by quantitative RT-PCR in tracheas. Data analyzed by ANOVA and Tukey posttest (boxes: 25^{th} percentile, median, 75^{th} percentile; whiskers: minimum and maximum). Different letters indicate significant differences at P<0.05.

FIG. 4A-B. Tracheal histomorphometry and histopathology of chickens treated as described in legend of FIG. 2. UV/UC=unvaccinated/unchallenged and UV/C=unvaccinated/challenged controls. Tracheal histomorphometry (A) mucosal thickness and (B) lymphocytic infiltration presented in arbitrary units using ImageJ. Values analyzed by ANOVA and Tukey posttest (boxes: 25^{th} percentile, median, 75^{th} percentile; whiskers: minimum and maximum). Different letters indicate significant differences at P<0.05.

FIG. 5A-C. Protection induced in chickens (n=20/group) by prime and booster vaccination regime with rLS/ArkSe.GMCSF and a commercial live-attenuated Massachusetts (Mass)-type vaccine. Chickens were prime vaccinated at 1 day of age with either 10⁶ EID₅₀/bird of rLS/Ark.Se.GM-CSF or a commercial live Mass vaccine at recommended dose. Booster vaccination was performed at 14 days of age. Mass/rLSArkSe.GMCSF=primed with Mass and boosted with recombinant virus. rLSArkSe.GMCSF/Mass=primed with recombinant virus and boosted with Mass. Controls include a group vaccinated with Mass only as well as UV/UC=unvaccinated/unchallenged and UV/C=unvaccinated/challenged. Challenge performed ocularly at 16 days post-boost with 10⁴ EID₅₀/bird of virulent Ark. Statistical analysis of respiratory signs and relative IBV RNA levels 5 days after challenge performed as described in the legend of FIG. 3. Different letters indicate significant differences at P<0.05.

FIG. 6A-C. Tracheal histomorphometry and histopathology of chickens treated as described in legend of FIG. 5. Tracheal histomorphometry: (A) mucosal thickness and (B) lymphocytic infiltration presented in arbitrary units using ImageJ were analyzed by ANOVA and Tukey posttest. (C) Necrosis scores analyzed by Kruskal-Wallis test and Dunn posttest. UV/UC=unvaccinated/unchallenged and UV/C=unvaccinated/challenged controls. Boxes: 25^{th} percentile, median, 75^{th} percentile; whiskers: minimum and maximum. Different letters indicate significant differences at P<0.05.

FIG. 7A-B. Ark Se antibody in chickens treated as described in the legend of FIG. 5. Ark Se antibody determined by ELISA using recombinant Ark Se protein coated plates. Data analyzed by ANOVA and Tukey posttest. Se antibody measured (A) 14 and (B) 22-days post boost. UV/UC=unvaccinated/unchallenged controls. Different letters indicate significant differences at P<0.05.

### DETAILED DESCRIPTION

Disclosed are recombinant Newcastle disease virus vectors expressing infectious bronchitis virus (IBV) spike ectodomain (Se) and granulocyte-macrophage colony-stimulating factor (GM-CSF), related polynucleotides, and methods of using the same for inducing an immune response against IBV, or vaccinating against IBV, which may be described herein using definitions as set forth below and throughout the application.

### Recombinant Newcastle Disease Virus (NDV) Vectors

Previously, the inventors disclosed a novel viral vector based on Newcastle disease virus LaSota strain that expressed a recombinant form of infectious bronchitis virus (IBV) surface glycoprotein (spike) ectodomain (rLS/ArkSe) and demonstrated that inoculation of subjects with said novel viral vector improved immune response to IBV challenge. See, U.S. Pat. No. 10,772,953, which is incorporated by reference herein in its entirety.

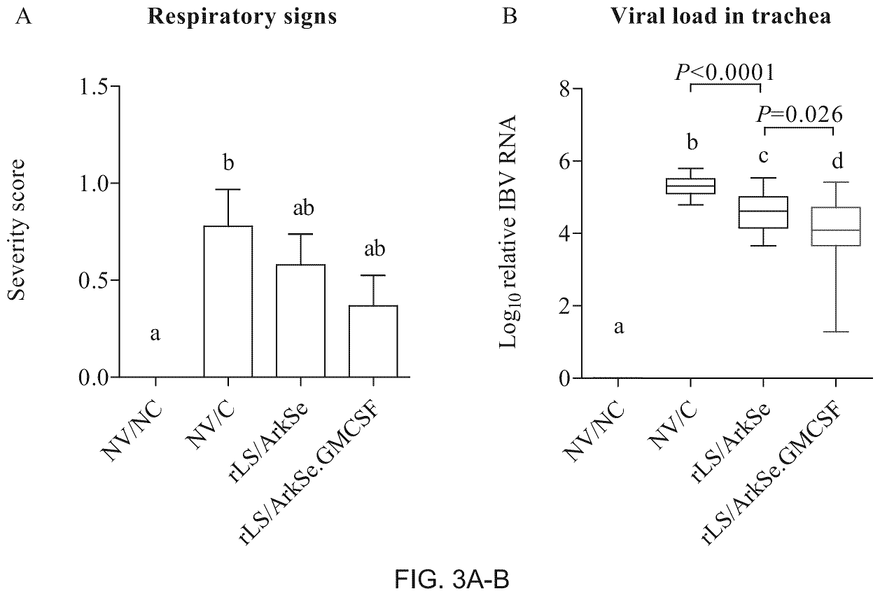

The inventors have discovered that a recombinant Newcastle disease virus vector expressing both IBV spike ectodomain (Se) and granulocyte-macrophage colony-stimulating factor (rLS/ArkSe.GMCSF) elicited a significantly enhanced immune response compared to the same vector lacking the capability of inducing GM-CSF expression (rLS/ArkSe), leading to improved protection of subjects to challenge by IBV (FIG. 3A-B).

Accordingly, in one aspect of the current disclosure, recombinant Newcastle disease virus vectors are provided. In some embodiments, the recombinant Newcastle disease virus vectors comprise: a nucleic acid encoding an infectious bronchitis virus (IBV) spike ectodomain (Se); and a nucleic acid encoding granulocyte-macrophage colony-stimulating factor (GM-CSF).

As used herein, “vector” refers to some means by which DNA or RNA can be introduced into a host. There are various types of vectors including virus, plasmid, bacteriophages, cosmids, and bacteria. As used herein, a “viral vector” or “Newcastle disease virus (NDV) vector” refers to recombinant virus, e.g., Newcastle disease virus, that has been engineered to express a heterologous polypeptide (e.g., a recombinant IBV Se protein and GM-CSF, as disclosed herein) in infected cells. The recombinant virus typically also includes cis-acting elements for expression of the heterologous polypeptide.

In some embodiments, GM-CSF is chicken (Gallus gallus) GM-CSF and has an amino acid sequence with at least 90%, at least 95%, or at least 98%, or 100% similarity to SEQ ID NO: 121:

PTTTYSCCYK VYTILEEITS HLESTAATAG LSSVPMDIRD KTCLRNNLKT FIESLKTNGT 60
EEESGIVFQL NRVHECERLF SNITPTPQVP DKECRTAQVS REKFKEALKT FFIYLSDVLP 120
EEKDCI 126

Referring now to the spike protein(S) of Infectious Bronchitis Virus (IBV), the S protein is expressed as a polypeptide having a length which typically is ˜1160-1170 amino acids depending on the particular variant of IBV. The S protein is expressed as a type I membrane protein. (See, e.g., Shen et al., Virology, 326 (2004) 288-298; and Winter et al., J. Virol., March 2008, p. 2765-2771; the contents of which are incorporated herein by reference in their entireties). The N-terminal amino sequence of the S protein (i.e., about amino acids 1-17) functions as a leader sequence, which directs the nascent S protein into the lumen of the endoplasmic reticulum (ER) and as such functions as a signal peptide. The signal peptide of the S protein is subsequently cleaved from the S protein to provide the N-terminus of the S1 domain (i.e., between amino acids 17 and 18). The S protein is cleaved again by furin endoprotease at a recognition site (RFRR/S) at about amino acid positions 534-538 to provide the C-terminus for the S1 subunit and to provide the N-terminus of the S2 subunit (i.e., between amino acids 537 and 538 in this example). The S2 subunit of the S protein includes a membrane anchor sequence at about amino acid positions 1096-1115 and a cytosolic portion from about amino acid positions 1116 to the C-terminus (i.e., to about amino acid position 1160-1170). The S1 subunit and the S2 subunit in the lumen of the ER associate together non-covalently to form the mature S protein. The mature S protein comprising the non-covalently associated S1 subunit and S2 subunit self-associates to form a multimeric structure, typically a trimeric structure (i.e., 3×(S1/S2). Subsequently, the S protein is transported to the surface of the cell where the S1 subunits and the N-terminal portions of the S2 subunits are expressed extracellularly and otherwise are referred to as the “ectodomain.” The membrane anchor sequences of the S2 subunits anchor the S protein in the cell membrane whereas and the C-terminal portions of the S2 subunits are expressed intracellularly.

The term “IBV” is meant to encompass numerous serotypes and strains of IBV that have been isolated and will be isolated in the future throughout the United States and the world and characterized, including but not limited to: B/D207/84; B/D274/84; B/UK167/84; B/UK142/86; E/D3896/84; E/UK123/82; Brazil/BR1/USP-73/09; 793B/4-91/91; FR/CR88121/88; China/Q1/98; China/LDL971/97; LX4; CAV/CAV9437/95; CAV/CAV1686/95; CAV/CAV56b/91; PA/Wolgemuth/98; PA/171/99; CA/557/03 S1; JAA/04 S1 vaccine; HN99 S1; N1/62/S1; GA08; Ark/ArkDPI/81 S1; Ark/Ark99/73; CAL99; CAL99/CAL99/99 S1; CAL99/NE15172/95 S1; Holte/Holte/54; JMK/JMK/64; Gray/Gray/60; Iowa/Iowa609/56; Ca/1737/04; DMA/5642/06 S1; GA07/GA07/07; QX/QXIBV/99; Mass/H52; Mass/H120; Mass/Mass41/41; Conn/Conn46/51 vaccine; FL/FL18288/71; DE/DE072/92 vaccine; Georgia 98; GA98/0470/98; GA-08; GA-13; and Dutch/D1466/81. The complete genomic sequences of many strains of IBV have been reported. (See Ammayappan et al., Virology Journal 2008, 5:157, reporting the genomic sequence of Ark/ArkDPI/81; which is incorporated herein by reference in its entirety). Live attenuated strains of IBV are available commercially as vaccines and may include Mass/Mass41/41 S1 and Ark/ArkDPI/81 S1.

There are numerous strains of infectious bronchitis virus. However, as discussed above, each strain has the common feature of an S protein that has two subunits that are proteolytically cleaved in a cell. In the disclosed working examples, the inventors disclose a recombinant Newcastle disease virus vector expressing the S protein ectodomain (Se) from the Arkansas strain of IBV. However, it is to be understood that the current disclosure contemplates the use of spike protein ectodomains from other strains of IBV. Moreover, such approaches may be advantageous to generate viral vectors expressing spike ectodomains from locally significant strains of IBV to better elicit relevant immune responses in subjects.

The native Se from IBV is proteolytically cleaved into two subunits which associate non-covalently and exist as trimers. Therefore, in some embodiments, the Se of the instant disclosure comprise a multimerization domain, e.g., a heterologous multimerization domain, which, in some embodiments, is SEQ ID NO: 122:

RMKQIEDKIE EIESKQKKIE NEIARIKKLV PRGSLE 36
or
SEQ ID NO: 123:
RMKQIEDKIE EILSKIYHIE NEIARIKKLI GER 33.

In some embodiments, the multimerization domain is an amino acid sequence that self-associates. In some embodiments, the multimerization domain comprises a trimerization motif as known in the art and the recombinant protein forms trimers in aqueous solution. (See, e.g., Kammerer et al., PNAS USA, Sep. 27, 2005, vol. 102, no. 39, pages 13891-13896; Kammerer et al., PLoSONE, August 2012, Volume 7, Issue 8, e43603; Sliepen et al., J. Biol. Chem., Volume 290, Number 12, Mar. 20, 2015, 7436-7442; Alvarez-Cienfuegos et al., Scientific Reports, 6:28643, 1-14; the contents of each of which are incorporated by reference in their entireties).

In some embodiments, the recombinant Se may be represented by Formula 1:

Nₜₑᵣ—SP-S1-Spacer-S2_{ecto}-MD-Cₜₑᵣ

In formula 1, “Nₜₑᵣ” represents the N-terminus of the protein and “Cₜₑᵣ” represents the C-terminus of the protein. As discussed herein, the N-terminus of the protein may be modified to include a non-naturally occurring moiety, such as an N-terminal acetyl group, and the C-terminus of the protein may be modified to include a non-naturally occurring moiety, such as an amide group.

In formula 1, “S1” represents the S1 domain of a spike protein(S) of IBV or a variant thereof. In some embodiments, S1 comprises an amino acid sequence of any of SEQ ID NOs: 3, 11, 19, 27, 35, 43, 51, 59, 67, 75, 83, 91, 99, 106, 110, 114, and 118 or an amino acid sequence having at least about 40%, 45%, 50%, 55%, 60%, 65%, 70%, 75%, 80%, 85%, 90%, 95%, 96%, 97%, 98%, or 99% sequence identity to any of SEQ ID NOs: 3, 11, 19, 27, 35, 43, 51, 59, 67, 75, 83, 91, 99, 106, 110, 114, and 118.

In formula 1, “S2_{ecto}” represents the ecto-subdomain portion of the S2 domain of the spike protein(S) or a variant thereof (i.e., the extracellular portion of the S2 domain or a variant thereof). In some embodiments, S2_{ecto} comprises an amino acid sequence of any of SEQ ID NOs: 5, 13, 21, 29, 37, 45, 53, 61, 69, 77, 85, 93, 101, 107, 111, 115, and 119 or an amino acid sequence having at least about 40%, 45%, 50%, 55%, 60%, 65%, 70%, 75%, 80%, 85%, 90%, 95%, 96%, 97%, 98%, or 99% sequence identity to any of SEQ ID NOs: 5, 13, 21, 29, 37, 45, 53, 61, 69, 77, 85, 93, 101, 107, 111, 115, and 119.

In formula 1, “Spacer” represents an optional spacer sequence of amino acids between S1 and S2_{ecto}. The Spacer typically does not comprise the amino acid sequence Arg-X-(Arg/Lys)-Arg. The amino acid sequence Arg-X-(Arg/Lys)-Arg-Ser is the recognition sequence for the furin endoprotease which cleaves between the arginine and serine residues where the serine residue provides the N-terminus for the S2 subunit. The amino acid sequence Arg-X-(Arg/Lys)-Arg-Ser is normally present in the native amino acid sequence of the S protein between the S1 domain and the S2 domain at about amino acid numbers 534-537 and is cleaved by the furin endoprotease during natural maturation of the S protein as discussed herein. Because the amino acid sequence Arg-X-(Arg/Lys)-Arg is not present in the Spacer of the recombinant protein, S1 and S2 remain covalently linked by the Spacer in the recombinant S protein. In the disclosed recombinant proteins, the native spacer between the S1 domain and the S2 domain may be replaced and/or mutated so as not to contain the amino acid sequence Arg-X-(Arg/Lys)-Arg.

In some IBV strains, the S2 ectodomain includes an additional recognition sequence for the furin endoprotease at about amino acid numbers 687-691 (e.g., Arg-Arg-Lys-Arg-Ser). (See, e.g., Yamada et al., J. Virol., September 2009, p. 8744-8758; the content of which is incorporated herein by reference in its entirety). Therefore, preferably in the disclosed recombinant proteins, the recombinant protein does not include the sequence Arg-X-(Arg/Lys)-Arg anywhere in the amino acid sequence of the recombinant protein. For recombinant S proteins derived from strains of IBV that include the additional recognition sequence for the furin endoprotease at about amino acid numbers 687-691, the amino acid sequence of the S2 ectodomain here may be replaced and/or mutated so as not to contain the amino acid sequence Arg-X-(Arg/Lys)-Arg.

In some embodiments of the disclosed recombinant proteins, the Spacer sequence may be relatively flexible, for example, so as to permit S1 and S2_{ecto} to mimic their natural interaction in the S protein. In some embodiments, the Spacer sequence is of sufficient length to permit the domains to mimic their natural interaction in the S protein. Suitable spacer sequences may include, but are not limited to, amino acid sequences of 3, 4, 5, 6, 7, 8, 9, 10, 11, 12, 13, 14, 15, 20, 25, 30, 35, 40, 45, or 50 amino acids or more, or a range bounded by any of these values (e.g., a spacer of 3-15 amino acids). In some embodiments, the Spacer sequence includes amino acids that provide flexibility to the Spacer and/or are small, neutral amino acids. In some embodiments, the spacer sequence comprises only glycine and/or serine residues or is rich in glycine and/or serine residues. For example, in some embodiments, the spacer sequence comprises at least about 50% glycine and/or serine residues, or at least about 55%, 60%, 65%, 70%, 75%, 80%, 85%, 90%, 95%, 96%, 97%, 98%, or 99% glycine and/or serine residues.

In formula 1, SP is a signal peptide. SP may comprise the native signal peptide of the S protein or a variant thereof. In some embodiments, SP comprises an amino acid sequence of any of SEQ ID NOs: 2, 10, 18, 26, 34, 42, 50, 58, 66, 74, 82, 90, 105, 109, 113, or 117 or an amino acid sequence having at least about 50%, 60%, 70%, 80%, 90%, or 95% sequence identity to any of SEQ ID NOs: 2, 10, 18, 26, 34, 42, 50, 58, 66, 74, 82, 90, 105, 109, 113, or 117.

In formula 1, SP may comprise a non-native signal peptide of the S protein (i.e., a heterologous signal peptide relative to the S protein). Signal peptides are known in the art. The core of the signal peptide contains a long stretch of hydrophobic amino acids (about 5-16 residues long) that has a tendency to form a single alpha-helix and is also referred to as the “h-region.” Signal peptides may begin with a short positively charged stretch of amino acids, which may help to enforce proper topology of the polypeptide during translocation by what is known as the positive-inside rule. Because of its close location to the N-terminus this short positively charged stretch of amino acid is called the “n-region.” At the end of the signal peptide there is typically a stretch of amino acids that is recognized and cleaved by signal peptidase and therefore named the “cleavage site.” A database of signal peptides is provided at Signal Peptide Website.

In some embodiments, the amino acid sequence of the IBV spike ectodomain is SEQ ID NO: 120, or an amino acid sequence at least 90% similar to SEQ ID NO: 120.

### Polynucleotides

The inventors used a reverse genetics system to generate the recombinant Newcastle disease virus vectors of the instant disclosure by expressing a polynucleotide comprising nucleic acids encoding Newcastle disease virus (NDV) proteins as previously described. See, for example, Estevez C., et al. Evaluation of Newcastle disease virus chimeras expressing the Hemagglutinin-Neuraminidase protein of velogenic strains in the context of a mesogenic recombinant virus backbone, Virus Res. 129:182-190; 2007, which is incorporated by reference herein in its entirety.

Accordingly, in another aspect of the current disclosure, polynucleotides are provided. In some embodiments, the polynucleotides comprise: (i) a nucleic acid encoding recombinant Newcastle disease virus; (ii) a nucleic acid encoding infectious bronchitis virus (IBV) spike ectodomain (Se); and (iii) a nucleic acid encoding granulocyte-macrophage colony-stimulating factor (GM-CSF); wherein (i)-(iii) are operably linked to one or more promoters.

Suitable promoters to express NDV in a reverse genetics system are known in the art and include, but are not limited to, T7 bacteriophage promoter. Thus, in such a system, cells comprising the disclosed polynucleotides should also comprise T7 bacteriophage polymerase to initiate transcription of NDV genes encoded by the disclosed polynucleotides.

In some embodiments, the nucleic acid encoding GM-CSF encodes the amino acid sequence SEQ ID NO: 121, or an amino acid sequence with at least 90% similarity to SEQ ID NO: 121. In some embodiments, the spike ectodomain (Se) is derived from Arkansas strain IBV. In some embodiments, the Se comprises a multimerization domain. In some embodiments, the multimerization domain is a heterologous multimerization domain. In some embodiments, the multimerization domain is SEQ ID NO: 122. In some embodiments, the recombinant Newcastle disease virus is LaSota strain of Newcastle disease virus. In some embodiments, the amino acid sequence of the Se is SEQ ID NO: 120. In some embodiments, the polynucleotides have the nucleic acid sequence SEQ ID NO: 124.

### Pharmaceutical Compositions and Vaccines

The compositions disclosed herein may include pharmaceutical compositions such as vaccine compositions comprising the presently disclosed recombinant vectors, which are formulated for administration to a subject in need thereof. Such compositions can be formulated and/or administered in dosages and by techniques well known to those skilled in the medical arts taking into consideration such factors as the age of the particular subjects and the route of administration.

Accordingly, in another aspect of the current disclosure, pharmaceutical compositions are provided. In some embodiments, the pharmaceutical compositions comprise recombinant Newcastle disease virus vector comprising: a nucleic acid encoding an infectious bronchitis virus (IBV) spike ectodomain (Se); and a nucleic acid encoding granulocyte-macrophage colony-stimulating factor (GM-CSF) and a pharmaceutically acceptable carrier.

The disclosed compositions may include additional components such as carriers, diluents, excipients, and surfactants, as known in the art. Further, the compositions may include preservatives (e.g., anti-microbial or anti-bacterial agents such as benzalkonium chloride). The compositions also may include buffering agents (e.g., in order to maintain the pH of the composition between 6.5 and 7.5).

The disclosed compositions may be administered as a vaccine in an amount sufficient to induce an immune response for protecting against infection. Inducing a protective response may include inducing sterilizing immunity against a pathogen (e.g., against IBV), or reducing the effects of the pathogen.

The compositions disclosed herein may be delivered via a variety of routes. Typical delivery routes include parenteral administration (e.g., intradermal, intramuscular, intraperitoneal, or subcutaneous delivery), intranasal, oral, and ocular (via eyedrop). Another route of administration may include in ovo administration (e.g., in ovo vaccination). Formulations of the pharmaceutical compositions may include liquids (e.g., solutions and emulsions), sprays, and aerosols.

The compositions disclosed herein may be co-administered or sequentially administered with other immunological, antigenic or vaccine or therapeutic compositions, including an adjuvant, or a chemical or biological agent given in combination with an antigen to enhance immunogenicity of the antigen.

### Methods of Using the Disclosed Recombinant Newcastle Disease Virus (NDV) Vectors

As described above, the inventors discovered that administration of recombinant NDV vector expressing IBV spike ectodomain (Se) and granulocyte-macrophage colony-stimulating factor (GM-CSF) elicited a protective immune response against subsequent challenge by IBV (FIG. 3A-B).

Accordingly, in another aspect of the current disclosure, methods of eliciting an immune response against IBV are provided. In some embodiments, the methods comprise administering an effective amount of a pharmaceutical composition comprising a recombinant Newcastle disease virus vector comprising: a nucleic acid encoding an infectious bronchitis virus (IBV) spike ectodomain (Se); and a nucleic acid encoding granulocyte-macrophage colony-stimulating factor (GM-CSF); and a pharmaceutically acceptable carrier, to a subject to elicit an immune response against IBV.

In another aspect of the current disclosure, methods of vaccinating a subject against IBV are provided. In some embodiments, the methods comprise administering an effective amount of a pharmaceutical composition comprising a recombinant Newcastle disease virus vector comprising: a nucleic acid encoding an infectious bronchitis virus (IBV) spike ectodomain (Se); and a nucleic acid encoding granulocyte-macrophage colony-stimulating factor (GM-CSF); and a pharmaceutically acceptable carrier, to a subject to elicit an immune response against IBV.

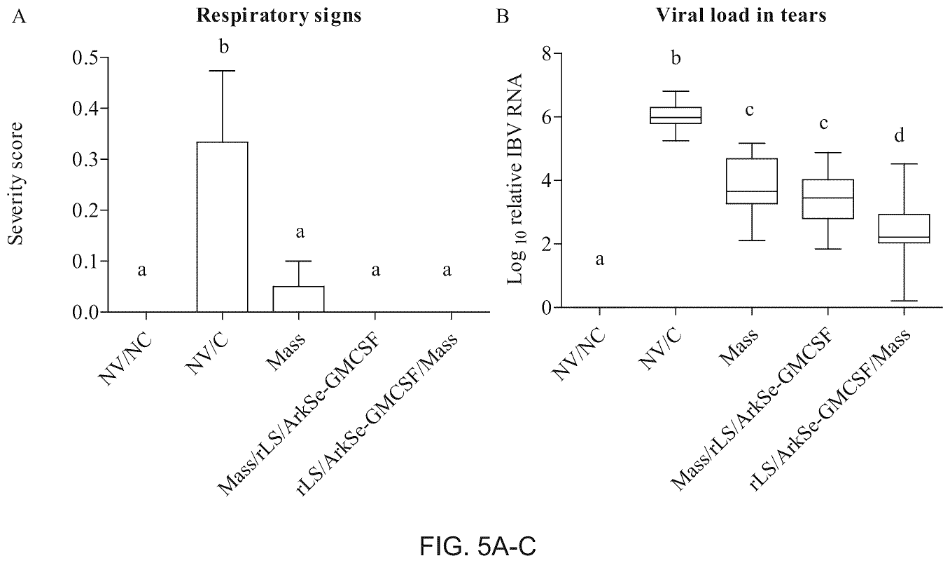

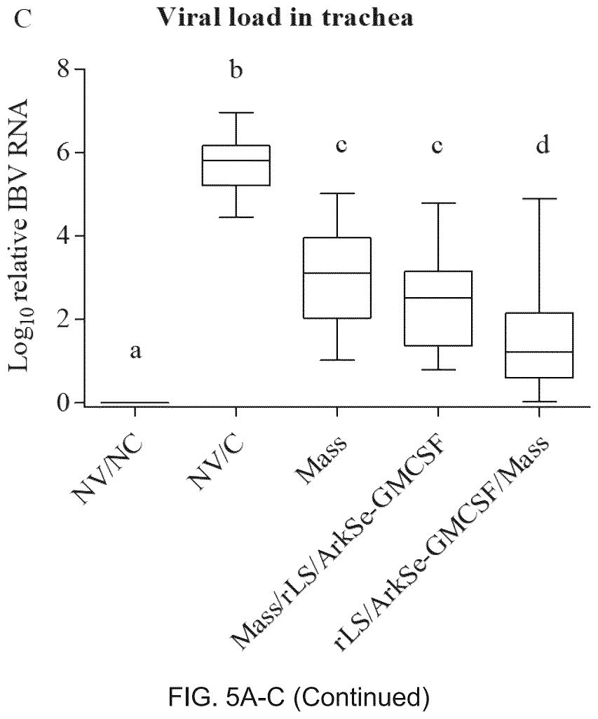

In addition, the inventors also discovered that a “prime-boost” strategy of administering the disclosed recombinant NDV vector expressing GM-CSF first, then subsequently administering a live-attenuated IBV vaccine, e.g., a Mass strain live-attenuated IBV vaccine, improved protection compared to administration of IBV live-attenuated vaccine alone, or administration of IBV live-attenuated vaccine and subsequent administration of the disclosed recombinant NDV vector expressing GM-CSF (FIG. 5A-C).

As used herein, a “prime-boost vaccination regimen” refers to a regimen in which a subject is administered a first composition and then after a determined period of time, the subject is administered a second composition, which may be the same or different than the first composition. The first composition (and the second composition) may be administered one or more times. The disclosed methods may include priming a subject with a first composition by administering the first composition at least one time, allowing a predetermined length of time to pass, and then boosting by administering the same composition or a second, different composition.

Accordingly, in some embodiments the methods of vaccinating a subject against IBV and the methods of eliciting an immune response against IBV further comprise administering a live-attenuated IBV vaccine, e.g., a Mass strain live-attenuated IBV vaccine, which is commercially available, to a subject. In some embodiments, the live-attenuated IBV vaccine is administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 5 weeks, 6 weeks, 7 weeks, 8 weeks, or more after administration of the disclosed recombinant DNV vectors.

The pharmaceutical compositions disclosed herein may be delivered to subjects at risk for acquiring an infection by IBV. In order to assess the efficacy of an administered immunogenic composition or vaccine, the immune response can be assessed by measuring the induction of antibodies to particular epitopes of IBV and/or cell-mediated responses against IBV. Antibody responses may be measured by assays known in the art such as ELISA. Immune responses also may be characterized by physiological responses. (See Li et al., Vaccine 28 (2010) 1598-1605; and Stemke-Hale et al., Vaccine 2005 Apr. 27; 23 (23): 3016-25, the content of which are incorporated herein by reference in their entireties.) Immune response also may be measured by reduction in pathological responses such as respiratory signs after challenge with IBV, or reduction in titer or load as measured using methods in the art including methods that detect nucleic acid of the pathogen. (See, e.g., U.S. Pat. No. 7,252,937, the content of which is incorporated by reference in its entirety). Immune response also may be measured by reduction in pathological responses such as a pathological response for an organ of the animal (e.g., the trachea) after challenge with IBV.

As used herein, “eliciting an immune response” refers to generation of an immune response against a particular antigen to which a subject has been exposed or administered, e.g., IBV spike ectodomain (Se), for example, by way of a viral vector expressing the antigen.

As used herein, an “immune response” may include an antibody response (i.e., a humoral response), where an immunized individual is induced to produce antibodies against an administered antigen (e.g., IgG (IgY), IgA, IgM, or other antibody isotypes). As used herein, an “immune response” also may include a cell-mediated response, for example, a cytotoxic T-cell response against cells expressing foreign peptides derived from an administered antigen in the context of a major histocompatibility complex (MHC) class I molecule.

As used herein, “potentiating” or “enhancing” an immune response means increasing the magnitude and/or the breadth of the immune response. For example, the number of cells that recognize a particular epitope may be increased (“magnitude”) and/or the numbers of epitopes that are recognized may be increased (“breadth”).

As used herein, “viral load” is the amount of virus present in a sample from a subject infected with the virus. Viral load is also referred to as viral titer. Viral load can be measured in variety of standard ways including copy Equivalents of the viral RNA (vRNA) genome per milliliter individual sample (vRNA copy Eq/ml). This quantity may be determined by standard methods that include RT-PCR.

### Methods of Generating Recombinant Newcastle Disease Virus

As described above, the inventors used a reverse genetics system to rescue the disclosed recombinant NDV vectors. See, for example, Estevez, C., et al., supra. Accordingly, in another aspect of the current disclosure, methods of generating a recombinant NDV vector are provided. In some embodiments, the methods comprise: expressing a polynucleotide comprising (i) a nucleic acid encoding recombinant Newcastle disease virus; (ii) a nucleic acid encoding infectious bronchitis virus (IBV) spike ectodomain (Se); and (iii) a nucleic acid encoding granulocyte-macrophage colony-stimulating factor (GM-CSF); wherein (i)-(iii) are operably linked to one or more promoters in a cell to generate a recombinant Newcastle disease virus vector.

Suitable cells for expression of recombinant NDV vectors include, but are not limited to, CEF cells, HEP2 cells, HEK293 cells, QM5 cells, BSRT7/5 cells, and BHK cells. Critically, the cells used to rescue the disclosed recombinant NDV vectors should express an appropriate DNA-dependent RNA polymerase that corresponds with the promoters contained in the disclosed polynucleotides, e.g., T7 polymerase and T7 promoters included in the disclosed polynucleotides.

Unless otherwise specified or indicated by context, the terms “a,” “an,” and “the,” mean “one or more.” For example, “protein” or “domain” should be interpreted to mean “one or more proteins” and “one or more domains,” respectively.

As used herein, “about”, “approximately,” “substantially,” and “significantly” will be understood by persons of ordinary skill in the art and will vary to some extent depending on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the terms “subject,” “host,” or “individual” typically refer to an animal at risk for acquiring an infection by infectious bronchitis virus (IBV), such as an avian species. The terms “subject,” “host,” or “individual” may be used interchangeably. Suitable avian species for the disclosed vaccine may include poultry such as members of the order Galliformes, and in particular the species Gallus gallus or the subspecies Gallus gallus domesticus.

### Polypeptide, Proteins, and Peptides

As used herein, polypeptide, proteins, and peptides comprise polymers of amino acids, otherwise referred to as “amino acid sequences.” A polypeptide or protein is typically of length ≥100 amino acids (Garrett & Grisham, Biochemistry, 2^{nd} edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2^{nd} edition, 1999, Brooks/Cole, 110). However, the terms “polypeptide,” “protein,” and “peptide” may be used interchangeably herein.

As contemplated herein, a polypeptide, protein, or peptide may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, or glycosylation (e.g., the addition of a glycosyl group to either asparagine, hydroxylysine, serine, or threonine, resulting in a glycoprotein). Additional modifications may include, but are not limited to polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine). In particular, the S protein of IBV generated in eukaryotic cells is heavily glycosylated. In some embodiments, the disclosed recombinant proteins of IBV may be modified to include a non-naturally occurring N-terminal modification such as an acetylation. In some embodiments, the disclosed recombinant proteins of IBV may be modified to include a non-naturally occurring C-terminal modification such as an amidation.

The amino acid sequences contemplated herein may include one or more amino acid substitutions relative to a reference amino acid sequence (e.g., relative to any of SEQ ID NOs: 1-119). In some cases, these substitutions may be conservative amino acid substitutions relative to the reference amino acid sequence. For example, a variant, mutant, or derivative polypeptide may include conservative amino acid substitutions and/or non-conservative amino acid substitutions relative to a reference polypeptide, which may include but is not limited to any of SEQ ID NOs: 1-119. “Conservative amino acid substitutions” are those substitutions that are predicted to interfere least with the properties of the reference polypeptide. In other words, conservative amino acid substitutions substantially conserve the structure and the function of the reference protein. Table 1 provides a list of exemplary conservative amino acid substitutions.

TABLE 1

| Original | Conservative |
|---|---|
| Residue | Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. In contrast, non-conservative amino acid substitutions generally disrupt and/or alter (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in a reference amino acid sequence (e.g., any of SEQ ID NOs: 1-119) that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant” of a reference polypeptide sequence may include a deletion relative to the reference polypeptide sequence. For example, SEQ ID NO: 3 (amino acids 18-532) and SEQ ID NO:5 (amino acids 538-1091) include N-terminal deletions and C-terminal deletions relative to reference sequence SEQ ID NO:1 (amino acids 1-1162).

The disclosed recombinant IBV S protein may include an N-terminal methionine residue that does not occur naturally in the native amino acid of the protein. For example, the amino acid sequence of recombinant IBV S proteins contemplated herein may include an N-terminal deletion relative to the amino acid sequence of the full-length S protein, and further, may be modified to include an N-terminal methionine residue that is not present in the amino acid sequence of the full-length S protein. For example a recombinant IBV S protein may include a deletion of amino acids 1-17 which are replaced with an N-terminal methionine.

The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues or a range of amino acid residues bounded by any of these values (e.g., an insertion or addition of 5-10 amino acids). A “variant” of a reference polypeptide sequence may include an insertion or addition relative to the reference polypeptide sequence.

A “fusion polypeptide” refers to a polypeptide comprising at the N-terminus, the C-terminus, or at both termini of its amino acid sequence a heterologous amino acid sequence, for example, a heterologous amino acid sequence that extends the half-life of the fusion polypeptide in serum. A “variant” of a reference polypeptide sequence may include a fusion polypeptide comprising the reference polypeptide. In some embodiments, the disclosed recombinant S IBV proteins may be defined as fusion polypeptides that include IBV amino acid sequences optionally fused to non-IBV amino acid sequences heterologous amino acid sequences (i.e., heterologous amino acid sequences).

A “fragment” is a portion of an amino acid sequence that is identical in sequence to but shorter in length than a reference sequence (e.g., any of SEQ ID NOs: 1-191). A fragment may comprise up to the entire length of the reference sequence, minus at least one amino acid residue. For example, a fragment may comprise from 5 to 1000 contiguous amino acid residues of a reference polypeptide. In some embodiments, a fragment may comprise at least 5, 10, 15, 20, 25, 30, 40, 50, 60, 70, 80, 90, 100, 150, 250, 300, 350, 400, 450, 500, 550, 600, 650, 700, 750, 800, 850, 900, 950, or 1000 contiguous amino acid residues of a reference polypeptide; or a fragment may comprise no more than 5, 10, 15, 20, 25, 30, 40, 50, 60, 70, 80, 90, 100, 150, 250, 300, 350, 400, 450, 500, 550, 600, 650, 700, 750, 800, 850, 900, 950, or 1000 contiguous amino acid residues of a reference polypeptide; or a fragment may comprise a range of contiguous amino acid residues of a reference polypeptide bounded by any of these values (e.g., 400-600 contiguous amino acid residues). Fragments may be preferentially selected from certain regions of a molecule. The term “at least a fragment” encompasses the full-length polypeptide. A “variant” of a reference polypeptide sequence may include a fragment of the reference polypeptide sequence. For example, SEQ ID NO:3 (amino acids 18-532) and SEQ ID NO:5 (amino acids 538-1091) comprise fragments of reference sequence SEQ ID NO:1 (amino acids 1-1162).

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of amino acid residue matches between at least two polypeptide sequences aligned using a standardized algorithm. Methods of polypeptide sequence alignment are well-known. Some alignment methods take into account conservative amino acid substitutions, non-conservative amino acid substitutions, deletions, and/or insertions. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” which is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence as defined by a particular SEQ ID number, for instance, a fragment of at least 15, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 contiguous amino acid residues of any of SEQ ID NOs: 1-119; or a fragment of no more than 15, 20, 30, 40, 50, 100, 150, 200, 250, 300, 350, 400, 450, 500, 550, 600, 650, at least 700, at least 750, at least 800, at least 850, at least 900, at least 950, or at least 1000 contiguous amino acid residues of any of SEQ ID NOs: 1-119; or over a range bounded by any of these values (e.g., a range of 500-600 amino acid residues). Such lengths are exemplary only, and it is understood that any fragment length supported by the sequences shown herein, in the tables, figures or Sequence Listing, may be used to describe a length over which percentage identity may be measured.

In some embodiments, a “variant” of a particular polypeptide sequence may be defined as a polypeptide sequence having at least 20% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In other embodiments, a pair of polypeptides may show, for example, at least 20%, at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% or greater sequence identity over a certain defined length of one of the polypeptides, or range of percentage identity bounded by any of these values (e.g., range of percentage identity of 80-99%).

The disclosed variants and mutants of a reference polypeptide may possess one or more biological activities associated with the reference polypeptide, or alternatively, the disclosed variants and mutants of a reference polypeptide may lack one or more biological activities associated with the reference polypeptide. For example, the disclosed recombinant IBV spike proteins may possess one or more biological activities associated with the wild-type IBV spike protein, or the disclosed recombinant IBV spike proteins may lack one or more biological activities associated with the wild-type IBV spike protein.

### Polynucleotides, Nucleic Acid, and Nucleic Acid Sequences

The terms “polynucleotide,” “nucleic acid” and “nucleic acid sequence” refer to a polymer of DNA or RNA nucleotide of genomic or synthetic origin (which may be single-stranded or double-stranded and may represent the sense or the antisense strand). The polynucleotides contemplated herein may encode and may be utilized to express one or more IBV polypeptides such as the disclosed recombinant spike ectodomain proteins of IBV.

The terms “nucleic acid” and “oligonucleotide,” as used herein, refer to polydeoxyribonucleotides (containing 2-deoxy-ribose), polyribonucleotides (containing ribose), and to any other type of polynucleotide that is an N glycoside of a purine or pyrimidine base. As used herein, the terms “A,” “T,” “C”, “G” and “U” refer to adenine, thymine, cytosine, guanine, uracil as a nucleotide base, respectively. There is no intended distinction in length between the terms “nucleic acid,” “oligonucleotide,” and “polynucleotide,” and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs.

A “fragment” of a polynucleotide is a portion of a polynucleotide sequence which is identical in sequence to but shorter in length than a reference sequence. A fragment may comprise up to the entire length of the reference sequence, minus at least one nucleotide. For example, a fragment may comprise from 5 to 1000 contiguous nucleotides of a reference polynucleotide. In some embodiments, a fragment may comprise at least 5, 10, 15, 20, 25, 30, 40, 50, 60, 70, 80, 90, 100, 150, 250, or 500 contiguous nucleotides of a reference polynucleotide; in other embodiments a fragment may comprise no more than 5, 10, 15, 20, 25, 30, 40, 50, 60, 70, 80, 90, 100, 150, 250, or 500 contiguous nucleotides of a reference polynucleotide; in further embodiments a fragment may comprise a range of contiguous nucleotides of a reference polynucleotide bounded by any of the foregoing values (e.g. a fragment comprising 20-50 contiguous nucleotides of a reference polynucleotide). Fragments may be preferentially selected from certain regions of a molecule. The term “at least a fragment” encompasses the full-length polynucleotide. A “variant,” “mutant,” or “derivative” of a reference polynucleotide sequence may include a fragment of the reference polynucleotide sequence.

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

A “recombinant nucleic acid” is a sequence that is not naturally occurring or has a sequence that is made by an artificial combination of two or more otherwise separated segments of sequence. This artificial combination is often accomplished by chemical synthesis or by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The term “promoter” as used herein refers to a cis-acting DNA sequence that directs RNA polymerase and other trans-acting transcription factors to initiate RNA transcription from the DNA or RNA (in an RNA virus) template that includes the cis-acting DNA of RNA sequence. A promoter may be “operably linked” to a coding sequence meaning that the promoter promotes transcription of the coding sequence, for example, as part of a vector.

### EXAMPLES

The following examples are illustrative and are not intended to limit the disclosed subject matter.

### Example 1-Protection Against Infectious Bronchitis Virus by Spike Ectodomain Subunit Vaccine

### Introduction

Insertion of foreign genes in recombinant Newcastle disease virus (NDV) LaSota (rLS) exhibits multiple advantages for vaccination of commercial broilers including safety, stability, and suitability for mass-administration. At the same time, chickens vaccinated with rLS are protected against NDV (15, 16). Insertion of the IBV S gene into the rLS genome reduces the virus' virulence to the level of the mild NDV Hitchner B1 vaccine, thus allowing safe vaccination of young naïve chickens (10, 14). Although vaccination with IBV S expressed from rLS (rLS/IBV.S) has shown promising results, the conferred protection against IBV challenge is still suboptimal. Shirvani et al. (13) reported protection in chickens vaccinated with rLS/IBV.S but reduction in virus shedding when chickens were vaccinated at one-day of age was dependent on route of challenge. Similarly, single-dose vaccination in one-day-old SPF chickens with rLS expressing a codon optimized IBV S provided significant protection against clinical disease after IBV challenge but did not reduce tracheal virus shedding (12). We previously developed rLS expressing the IBV Ark-type S-ectodomain (rLS/ArkSe), that is, the S protein excluding the transmembrane anchor and short cytoplasmic domain of S2 that is not shown to the immune system during infection. Vaccination with a relatively high dose (10⁷ EID₅₀/bird) of rLS/ArkSe reduced signs and tracheal lesions in chickens but did not decrease the viral load in tear fluids of challenged chickens. The high dose required for protection also makes this vaccine unappealing for the industry.

Others have demonstrated that DNA vaccines co-administered with plasmid cytokine adjuvants increase humoral and cellular immune responses in vaccinated animals (17, 18, 19). The cytokine granulocyte-macrophage colony-stimulating factor (GMCSF) enhances immune responses by attracting macrophages and inducing their maturation, thus resulting in increased antigen presentation (20). Accumulating evidence indicates a strong effect on differentiation and maturation of dendritic cells as well as expression of MHC and co-stimulatory molecules (21, 22, 23, 24) resulting in enhancement of antigen-specific humoral and cellular immune responses. For example, simultaneous inoculation with plasmid DNA expressing GMCSF has been reported to increase protection against classical swine fever virus (25), herpes simplex virus (26), and foot-and-mouth disease virus (18). Prime and booster vaccination with both plasmids carrying the S1 gene of IBV and the chicken GMCSF gene have shown significant enhancement of humoral and cellular responses in chickens compared to vaccination with S1 plasmid alone (27). Recombinant human adenoviruses (rAd) expressing chicken GMCSF and the S1 gene of a nephropathogenic IBV strain have been shown to confer enhanced protection against homologous IBV challenge in chickens. Chickens vaccinated with rAd-S1 fused or co-administered with GMCSF showed reduced nephropathy and 100% protection compared to 70% protection in chickens vaccinated with rAd-S1 alone (28).

We developed rLS co-expressing the Se of an Arkansas (Ark)-type IBV and chicken GM-CSF (rLS/ArkSe.GMCSF). We initially compared the effectiveness of the construct containing GMCSF versus a previously produced construct expressing Se only (rLS/ArkSe). Because live vaccines belonging only to the Massachusetts (Mass) serotype have been licensed in most countries worldwide, in a second trial we explored enhancing cross-protection of an attenuated Mass vaccine in a prime-boost vaccination regime with rLS/ArkSe.GMCSF. If confirmed, this strategy would provide protection against regional IBV types using tailored rLS.

### Materials and Methods

Chickens. White leghorn chickens were hatched from specific pathogen-free (SPF) embryonated eggs (Wayward Acres, Pickens, SC) and maintained in Horsfall-type isolators in biosafety level 2 facilities. Experimental procedures and animal care were performed in compliance with all applicable federal and institutional animal care and use guidelines. Auburn University College of Veterinary Medicine is an Association for Assessment and Accreditation of Laboratory Animal Care (AAALAC International)-accredited institution.

Challenge virus. The previously characterized virulent Ark-type IBV strains with GenBank accessions #DQ458217 (29) and #JN861120 (30) were used in experiments 1 and 2 respectively. Challenged viruses were titered in SPF embryonated chicken eggs as accepted (31) with some modifications previously described (32).

Generation of rLS containing chicken GM-CSF and IBV Ark S-ectodomain (Ark.Se) genes. The previously generated pLS—I—RFP-GFP plasmid (33) was used as a backbone to clone the chicken GMCSF and IBV Se genes into the NDV LaSota genome through two-steps of cloning. First, a DNA fragment encoding the C-terminal 126 amino acids (aa) of the chicken GMCSF protein (19-144 aa, GenBank: GQ421598.1) was commercially synthesized with codon-optimization for chickens (GeneScript, Piscadaway, NJ, USA). The synthesized chicken GMCSF fragment was amplified by PCR with a pair of gene-specific primers and cloned into the pLS—I—RFP-GFP vector to replace the RFP gene using an In-Fusion® PCR Cloning Kit following the manufacturer's instructions (Clontech, Mountain View, CA, USA). Secondly, the resulting plasmid, pLS-GMCSF-GFP, was used as a vector to clone the IBV Se gene into the LaSota genome. The Ark-type IBV S-ectodomain (S1+S2 lacking the transmembrane domain and cytoplasmic tail) gene was amplified by PCR with a pair of gene-specific primers from a previously generated plasmid pLS/ArkSe (14) and cloned into the pLS-GMCSF-GFP vector to replace the GFP gene using the In-Fusion® PCR Cloning Kit. The sequences of the primers used for the foreign gene amplification and the In-fusion PCR cloning in this study are available upon request. The final resulting plasmid, pLS-GMCSF-ArkSe, was used to rescue the recombinant virus using the reverse genetic system as described previously (34). The rescued recombinant virus, designated as rLS/GMCSF/ArkSe, was propagated in chicken embryonated eggs two more times. The allantoic fluid harvested from infected embryos at the third egg passage was aliquoted and stored at −8° C. as a stock.

Virus titration, pathogenicity assessment, and sequence analysis. The rLS/ArkSe.GMCSF and the rLS virus stocks were titrated both by standard hemagglutinating activity (HA) test in a 96-well microplate and 50% egg infective dose (EID₅₀) determination in 9-day-old SPF chicken embryos (35). Pathogenicity of the viruses was assessed by standard procedures; i.e. mean death time (MDT) in chicken embryos and intracerebral pathogenicity index (ICPI) in one-day-old SPF chickens (35). The nucleotide sequences of the rLS/ArkSe.GMCSF virus were determined by sequencing the RT-PCR products amplified from the viral genome as described previously to confirm the sequence fidelity of the rescued virus (36).

Expression of the IBV S-ectodomain protein. DF-1 cells were infected with the rLS/ArkSe.GMCSF virus and examined by immunofluorescence assay (IFA) with a polyclonal anti-Ark-type IBV chicken serum as described previously (15) with minor modifications. An NDV-specific monoclonal antibody against the HN protein (kindly provided by Dr. Ron Iorio, University of Massachusetts Medical School) was included in the IFA to detect the NDV HN protein as an NDV infection control. After incubation with primary antibodies (anti-IBV and anti-NDV HN) and secondary antibody conjugates (Alexa Fluor® 568 conjugated goat anti-mouse IgG and FITC-labeled goat anti-chicken IgG), and washing with PBS, infected cells were stained with DAPI (300 nM) (Thermo Fisher Scientific, Waltham, MA) at room temperature for 5 min as an extra step of the IFA to show DF-1 cell nuclei. Cytopathic effect (CPE) and fluorescence images were monitored/photographed using an EVOS FL Cell Imaging System (Thermo Fisher Scientific, Waltham, MA) at 400× magnification. Green-, red-, and blue-fluorescence images taken from the same field of virus-infected cells were merged into a single image to examine the co-localization of the IBV Se and NDV HN proteins.

Experimental design. Two experiments were conducted to evaluate the extent of protection. In experiment 1, we compared protection conferred by single vaccination with rLS expressing Ark Se (rLS/Ark.Se) and rLS co-expressing Ark Se and GM-CSF (rLS/ArkSe.GMCSF). In experiment 2, we evaluated cross-protection induced by live Mass vaccination when administered in prime and boost regime with rLS/ArkSe.GMCSF.

Experiment 1. Protection conferred by single vaccination with rLS/ArkSe and rLS/ArkSe.GMCSF. Four groups of chickens (n=19/group) were established. Experimental groups 1 and 2 were vaccinated at 7 days of age (DOA) with 100 μl per bird containing 10⁶ 50% embryo infectious doses (EID₅₀) of rLS/ArkSe and rLS/ArkSe.GMCSF respectively. Control groups 3 and 4 included unvaccinated/challenged and unvaccinated/unchallenged chickens respectively. Blood was collected 23 days after vaccination for serum NDV antibody determination by hemagglutination inhibition test as accepted (37). At 31 days of age, chickens in groups 1-3 were individually challenged with 100 μl (25 μl in each nostril and each eye) containing 10⁴ EID₅₀/bird of Ark virulent virus stock (GenBank accession #DQ458217). Protection was evaluated five days after challenge by individual assessment of respiratory signs, tracheal histomorphometry, and viral load in tracheas of challenged chickens as previously described (14). In brief, nasal and/or tracheal rales were evaluated blindly by close listening to each bird and scored as 0 (absent), 1 (mild), 2 (moderate), or 3 (severe) and scoring data subsequently analyzed by Kruskal-Wallis test followed by Dunn post-test. Viral load in tracheas were determined by quantitative reverse transcriptase polymerase chain reaction (qRT-PCR). Briefly, IBV RNA was extracted from homogenized tracheal samples with TriReagent® RNA/DNA/protein isolation reagent (Molecular Research Center, Cincinnati, OH) as per the manufacturer's recommendations. Relative viral copies were determined by quantitation of viral RNA using TaqMan© qRT-PCR as described (38). Viral RNA data were analyzed by one-way ANOVA followed by Tukey multiple comparisons post-test. Differences were considered significant with P<0.05. Tracheal histopathology and histomorphometry was performed as described previously (29). Briefly, tracheal sections were routinely prepared for hematoxylin and eosin stain histopathological for evaluation. ImageJ software (https://imagej.nih.gov/ij/download.html) was used to measure the tracheal mucosal thickness and lymphocytic infiltration. Tracheal histomorphometry data (arbitrary units using ImageJ) were analyzed by one-way ANOVA followed by Tukey multiple comparison post-test.

Experiment 2. Cross-protection induced by prime-boost regime with live Mass vaccine and rLS/ArkSe.GMCSF. Chickens were divided into five groups. Groups 1 and 2, consisting of 28 birds each, were vaccinated with the recommended dose of an attenuated Mass vaccine (Pfizer, New York, NY) at 1 DOA. Group 2 was boosted 16 days after prime with 10⁶ EID₅₀ of rLS/ArkSe.GMCSF. Group 3 was primed at 1 DOA with 10⁶ EID₅₀ of rLS/ArkSe.GMCSF and boosted 16 days after prime with the commercial Mass vaccine. Groups 4 and 5 were unvaccinated/challenged (n=19) and unvaccinated/unchallenged (n=27) controls respectively. Sera were collected 14 days post-boost from all groups. Twenty chickens of groups 1, 2, 3, and 4 were challenged as described above with 10⁴ EID₅₀/bird of virulent Ark (GenBank accession #JN861120) at 33 DOA (i.e. 16 days after booster in groups 2, 3, and 4). Protection against challenge was evaluated 5 days post-challenge by clinical signs, viral load in trachea, and tracheal histomorphometry as described above. In addition, histopathology scoring of tracheal necrosis was performed. Finally, viral load was also determined in lachrymal fluids. IBV RNA was extracted from tear samples using the QIAmp Viral RNA Mini Kit (Qiagen, Valencia, CA) and qRT-PCR performed as described above. The remaining eight chickens of these groups were separated and remained unchallenged for antibody determinations at 22 days after boost.

Ark Se serum antibody. Ark Se specific antibody levels in sera were determined at 14 and 22 days after boost using a Se specific ELISA previously described (14). In brief, ELISA plates (Nunc MaxiSorp®, San Diego, CA) were coated overnight with 100 μl per well of 0.25 μg/ml soluble trimeric recombinant spike ectodomain protein. Plates were blocked with 200 μl/well of phosphate-buffered saline (PBS) containing 1% bovine serum albumin and 0.05% Tween 20. Individual chicken pre-challenge sera were diluted 1:100 in PBS and incubated in the wells for 30 minutes at room temperature. All following steps were performed with reagents of a commercial IBV ELISA kit (Idexx Laboratories Inc., Westbrook, ME) following the manufacturer's guidelines. Absorbance values were analyzed by ANOVA followed by Tukey post-test.

### Results

Biological properties of the rLS/ArkSe.GMCSF virus. As shown in Table 1, the rLS/ArkSe.GMCSF virus replicated in chicken embryos but achieved slightly lower HA and EID₅₀ titers compared to those of the LaSota strain. This result suggests that the relatively sizable foreign gene insertion (totally 3,840 nts, representing approximately 25% of the LaSota genome) likely influenced the virus' replicating ability. As reported previously, the recombinant virus showed low pathogenicity with a similar ICPI (0.18) to the LaSota virus (0.15) and higher MDT (>150 hrs) than the LaSota virus (134 hrs). The nucleotide sequence analysis revealed that the rLS/ArkSe.GMCSF virus maintained its sequence fidelity after three passages in chicken embryos. The nucleotide sequences of the chicken GMCSF gene (381 nts), the IBV Se gene (3,459 nts), and the complete genome of rLS/ArkSe.GMCSF (19,812 nts) are available upon request.

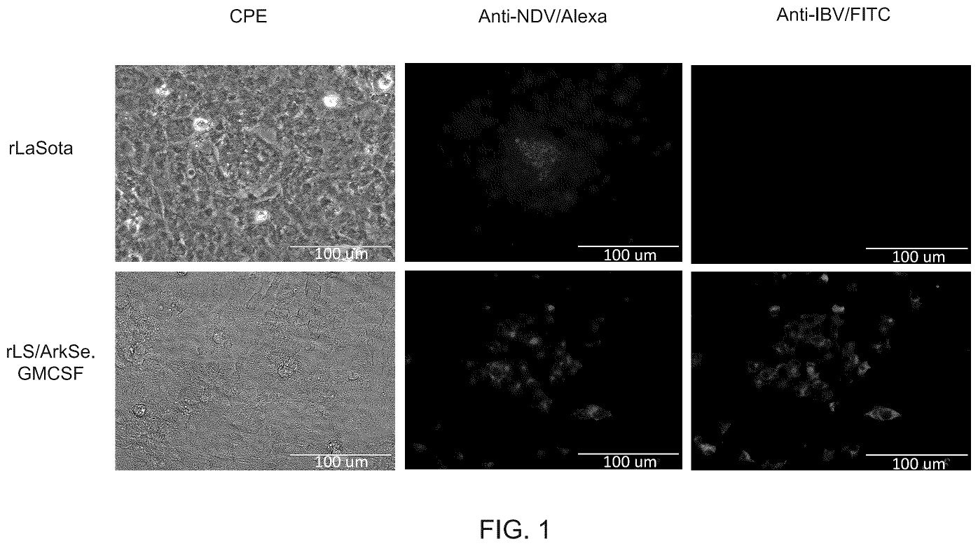

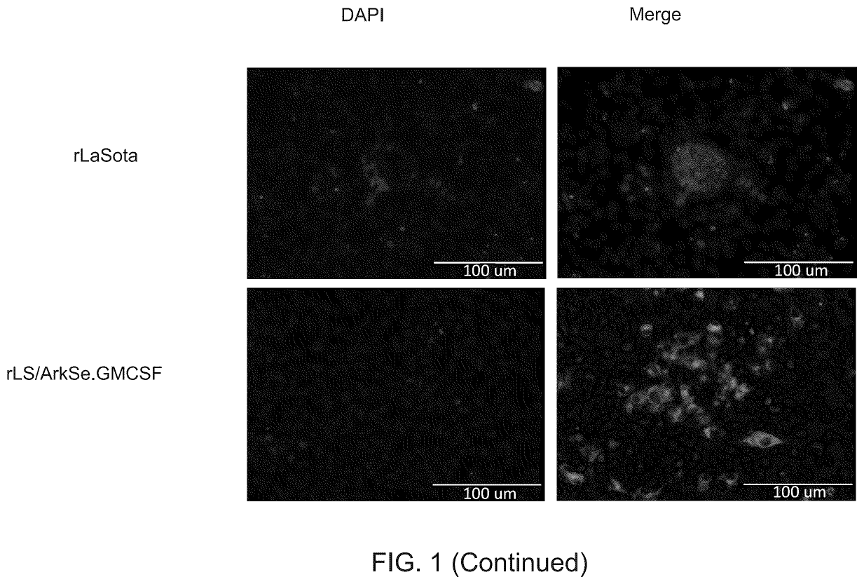

Expression of IBV Ark Se and NDV HN proteins in cells infected with the rLS/ArkSe.GMCSF virus. The red, green, and blue fluorescence shown in FIG. 1 represent the NDV HN protein, IBV S-ectodomain protein, and cell nuclei, respectively. The red fluorescence was observed in LS-infected cells, but no green fluorescence was detected in the same field of infected cells, demonstrating the antibodies' specificity. When examining rLS/ArkSe.GMCSF virus-infected DF-1 cells, both green and red fluorescence were observed, indicating expression of the IBV Se and NDV HN proteins. After merging fluorescent images that were taken in the same field, green and red fluorescence co-localized to the same infected cells. This result confirmed that the IBV Se protein was co-expressed with the NDV HN protein from the rLS/ArkSe.GMCSF virus-infected cells.

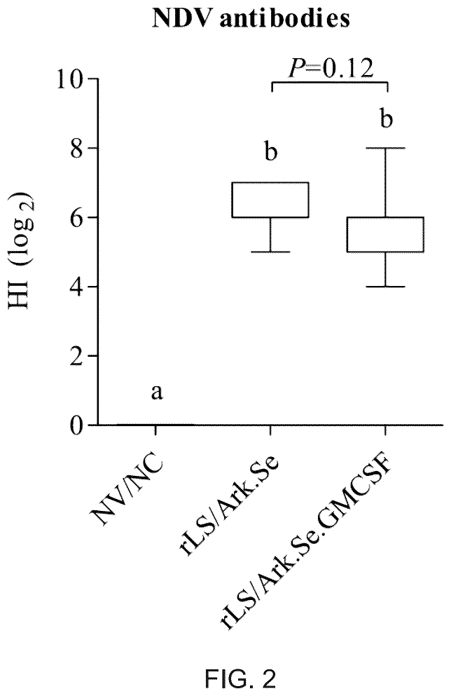

Experiment 1. The rLS/ArkSe.GMCSF vaccine was safe, as no adverse side effects were detected after vaccination in 7-day-old chickens. Both chicken groups receiving rLS showed NDV HI titers significantly higher than unvaccinated controls (FIG. 2). As seen in this figure, the group vaccinated with rLS not expressing GMCSF exhibited slightly higher average NDV HI titers compared to chickens vaccinated with rLS/ArkSe.GMCSF, but the difference did not achieve statistical significance.

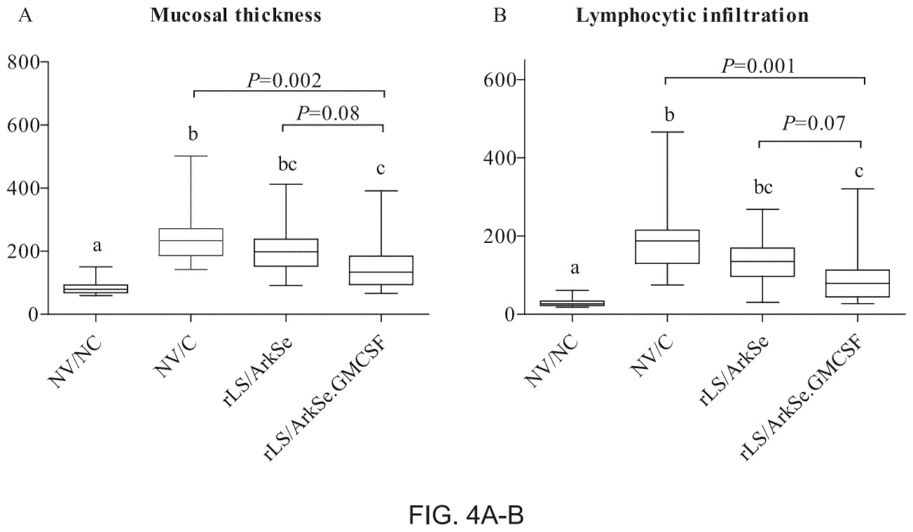

The parameters used herein to evaluate protection in this experiment showed improved protection by rLS co-expressing the IBV Se and the GMCSF cytokine. FIG. 3A shows mostly absence of respiratory signs in chickens vaccinated with rLS/ArkSe.GMCSF not differing significantly from unvaccinated/unchallenged controls. Consistently, viral IBV RNA in the trachea was also significantly lower (P<0.05) in this group (FIG. 3B). In contrast, chickens vaccinated with rLS/ArkSe as well as unvaccinated controls showed significantly higher (P<0.05) viral RNA in the trachea and increased respiratory signs after challenge. Tracheal histomorphometry showed a trend consistent with both respiratory signs and viral load. A significant reduction of mucosal thickness and lymphocytic infiltration was observed in chickens vaccinated with rLS/ArkSe.GMCSF compared to unvaccinated/challenged chickens (FIG. 4). In contrast, while a reduction of tracheal lesions was detected in rLS/ArkSe compared to unvaccinated/challenged birds, the difference did not achieve significance.

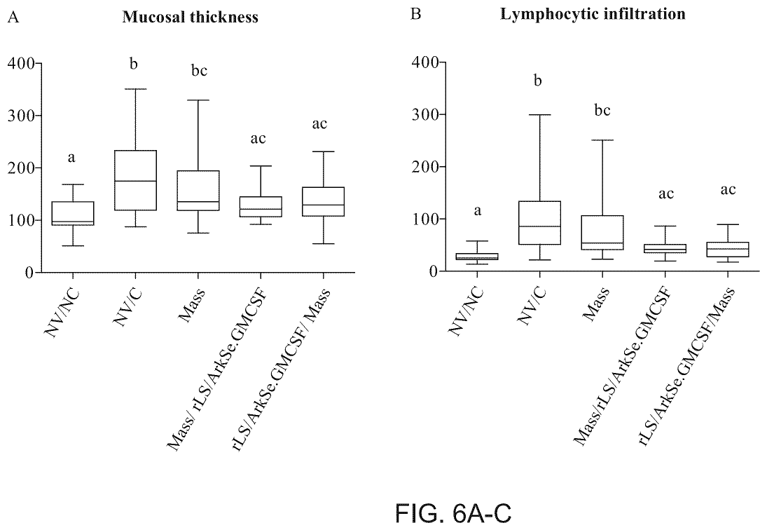

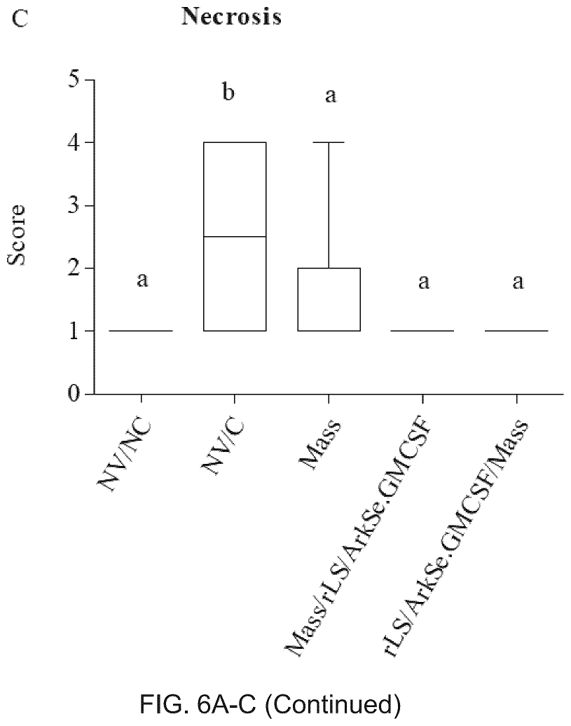

Experiment 2. As seen in FIG. 5, all vaccinated groups showed significantly lower respiratory signs, and viral loads in both tears and tracheas (P<0.05) than unvaccinated controls after virulent Ark challenge. Although the viral loads in tears and tracheas were reduced in all vaccinated groups, the group primed with rLS/ArkSe.GMCSF and boosted with Mass achieved maximum reduction of Ark IBV RNA. These levels were significantly lower (P<0.05) than chickens vaccinated with Mass only and chickens primed with Mass and boosted with rLS/ArkSe.GMCSF. When comparing the two latter groups, chickens primed with Mass and boosted with rLS/ArkSe.GMCSF showed lower values than Mass-only vaccinated chickens but without achieving statistical significance. The results of tracheal histomorphometry and histopathology in FIG. 6 showed prime and boost with rLS/ArkSe.GMCSF and Mass in either direction protected the tracheal mucosa effectively and significantly better than Mass alone against Ark challenge. Indeed, tracheal mucosal thickness, lymphocyte infiltration, and presence or absence of necrosis were not different (P<0.05) from unvaccinated/unchallenged controls. In contrast, chickens vaccinated only with Mass showed considerable tracheal damage compared to negative controls.

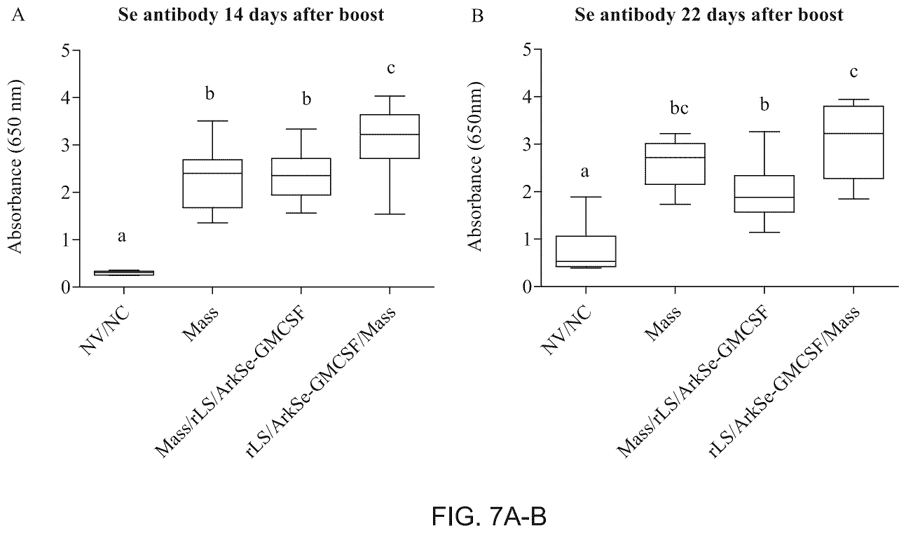

Se antibody levels in prime/boost chickens. Antibody levels detected 14 days after booster vaccination were highest (P<0.05) in the group primed with rLS/ArkSe.GMCSF and boosted with Mass compared to prime and boost in the other direction as well as Mass vaccination only (FIG. 7). Consistently, 22 days post-boost antibody levels were found to be significantly higher in the chickens primed with rLS/ArkSe.GMCSF and boosted with Mass compared to those primed with Mass and boosted with rLS/ArkSe.GMCSF. However, on day 22 antibody levels for the boosted groups were not significantly different when compared with chickens vaccinated with Mass only.

### DISCUSSION

The current results demonstrate that the recombinant LaSota vaccine construct expresses the IBV Se successfully in cell culture and is stable after passages in embryonated eggs. Corroborating previous results (14), although the replication rate is reduced compared to the parental LaSota strain, the low pathogenicity, as determined by both MDT and ICPI, allows safe vaccination of young chickens. Finally, the recombinant virus replicates well in chickens as it elicits antibody responses against NDV.

Breeder and layer hens are commonly vaccinated against NDV. In the southeastern U.S., some companies use up to four live NDV vaccines in broiler breeders until 16 weeks of age. In addition, some companies may vaccinate the progenies with recombinant HVT-NDV in ovo (Dr. J. Cline, Wayne Farms, personal communication). The presence of maternal NDV antibodies and/or antibodies resulting from active in ovo immunization may interfere with rLS vaccination in commercial settings. Indeed, others have shown that presence of NDV maternal antibodies in chickens at the time of vaccination with rNDV expressing avian influenza virus (AIV) proteins can prevent development of immunity from rNDV expressing the H5 of AIV (39). In addition, presence of maternal antibody to AIV has also been shown to interfere with active vaccination with NDV expressing the AIV hemagglutinin (40). We presently lack information on use of rLS expressing the IBV spike glycoprotein in chickens of commercial origin. The levels of antibodies in broilers certainly vary around the world depending on NDV pressure of infection and NDV vaccination programs used. Thus, varying levels of immunity against NDV will permit or impede the vaccine to break through immunity and replicate successfully at variable times after hatch. In addition, use of better adjuvants should also have an effect on the effectiveness of recombinant NDV vaccines in commercial chickens.

Several studies have shown that use of rLS expressing IBV S or subunits of S induce less than optimal protection against IBV virulent challenge (9, 10, 12, 13). Thus, improved adjuvants may be needed to achieve optimum protection. As discussed above, others have reported that both co-administration of subunit vaccines with GMCSF and DNA vaccines with GMCSF show significant improved immunogenicity. Similarly, co-expression of foreign proteins and GMCSF as well as co-administration of GMCSF with recombinant virus constructs have also shown enhanced protection (17, 18, 19, 25, 26, 27, 28). However, having to inject such vaccines individually in large broiler populations limits their applicability for the poultry industry. In the current experiments, the successful replication of the rLS after administration via a mucosal route indicates that mass administration via spray is feasible. Moreover, the current results demonstrate that insertion of GMCSF in rLS expressing an IBV spike-ectodomain enhances the effectiveness of vaccination after single and prime-booster vaccination. This result corroborates the results by others (discussed above) who have demonstrated enhanced protection when using GMCSF to accompany other vaccine settings.

IBV's extensive genotypic and phenotypic variability is the result of genetic diversity generated by mutations made by the viral RNA dependent RNA polymerase and by recombination events. The evolutionary process continues when abundant new variants serve as the material of selection (1). Thus, it has become clear that the use of live attenuated IBV vaccines may have solved the problem in the short term but has favored recombination events worsening the problem in the long term. In addition, because often more than one serotype is acting in a particular region, IBV prevention considers inclusion of various serotypes in vaccination programs. This practice augments the likelihood of recombination among diverse genotypes even more. Indeed, numerous outbreaks of disease are currently being caused by vaccine-like viruses and wild-vaccine recombinant viruses (5, 41). Mass-type live vaccine viruses, same as every other IB coronavirus, have the ability to recombine with other IBV types. After initial reports of protection conferred by Mass-type vaccines in the early 1960s, Mass-type vaccines were registered and licensed worldwide. During years thereafter, other type-specific live vaccines were developed to protect against regionally emergent serotypes. Pharmaceutical companies made efforts to sell these vaccines in other regions with variable success as countries became more careful at allowing the introduction of foreign IBV strains/genes into their industry. In fact, several countries still only allow Mass-type vaccines to be used. Thus, because Mass wild and vaccine viruses are endemic worldwide, a combination of live Mass with a recombinant vaccine virus that enhances its cross-protection provides an advancement towards a better solution to the problem. The current results demonstrate that use of rLS/ArkSe.GMCSF with Mass in a prime/boost vaccination regime enhances the cross-protection capability of Mass significantly, as this combination protected against Ark challenge. Interestingly, based on viral load and tracheal histopathology, better protection was achieved when priming was performed with rLS/ArkSe.GMCSF followed by Mass boosting. The inverse approach, i.e. prime with Mass and boost with rLS/ArkSe.GMCSF, provided similar protection of the tracheal epithelium but reduction of viral load was less effective. Higher levels of Ark Se specific antibodies were elicited by the former combination, which may explain those better results. However, the immunological mechanism behind this difference cannot be explained in the current study. We also analyzed antibody avidity of both groups but were not able to detect any differences (data not shown). Using Mass and replacing attenuated type-specific booster vaccines by tailored type-specific S-ectodomain expressed from recombinant virus provides an opportunity to achieve good protection, and equally important, should reduce potential recombination and emergence of new IBV variants.

### REFERENCES

1. Toro H, Jackwood M W, van Santen V L. Genetic diversity and selection regulates evolution of infectious bronchitis virus. Avian Dis. 56:449-455; 2012.
2. Gallardo R A, van Santen V L, Toro H. Host intraspatial selection of infectious bronchitis virus populations. Avian Dis. 54:807-813; 2010.
3. Mckinley E T, Hilt D A, Jackwood M W. Avian coronavirus infectious bronchitis attenuated live vaccines undergo selection of subpopulations and mutations following vaccination. Vaccine. 26:1274-1284; 2008.
4. van Santen V L, Toro H. Rapid selection in chickens of subpopulations within ArkDPI-derived infectious bronchitis virus vaccines. Avian Pathol. 37:293-306; 2008.
5. Jackwood M W, Lee D H. Different evolutionary trajectories of vaccine-controlled and non-controlled avian infectious bronchitis viruses in commercial poultry. PLOS ONE 12: e0176709; 2017.
6. Ghetas A M, Thaxton G E, Breedlove C, van Santen V L, Toro H. Effects of adaptation of infectious bronchitis virus Arkansas attenuated vaccine to embryonic kidney cells. Avian Dis. 59:106-113; 2015.
7. Toro H, Pennington D, R. A. Gallardo, van Santen V L, van Ginkel F W, Zhang J F, Joiner K S. Infectious bronchitis virus subpopulations in vaccinated chickens after challenge Avian Dis. 56:501-508; 2012.
8. Zegpi R A, Gulley S, van Santen V L, Joiner K S, Toro H. Infectious bronchitis virus vaccination at day one of age further limits cross-protection. Avian Dis. 63:302-309; 2019.
9. Toro H, J. F. Zhang, Gallardo R A, van Santen V L, van Ginkel F W, Joiner K S, Breedlove C. S1 of distinct IBV population expressed from recombinant adenovirus confers protection against challenge. Avian Dis. 58:211-215; 2014.
10. Toro H, Zhao W, Breedlove C, Zhang Z, van Santen V, Yu Q. Infectious bronchitis virus S2 expressed from recombinant virus confers broad protection against challenge. Avian Dis. 58:83-89; 2014.
11. Eldemery F, Li Y, Q. Yu, van Santen V L, Toro H. Infectious bronchitis virus S2 of 4/91 expressed from recombinant virus does not protect against Ark-type challenge. Avian Dis. 61:397-401; 2017.
12. Abozeid H H, Paldurai A, Varghese B P, Khattar S K, Afifi M A, Zouelfakkar S, El-Deeb A H, El-Kady M F, Samal S K. Development of a recombinant Newcastle disease virus-vectored vaccine for infectious bronchitis virus variant strains circulating in Egypt. Vet Res. 50:12; 2019.
13. Shirvani E, Paldurai A, Manoharan V K, Varghese B P, Samal S K. A Recombinant Newcastle Disease Virus (NDV) Expressing S Protein of Infectious Bronchitis Virus (IBV) Protects Chickens against IBV and NDV. Sci Rep. 8:11951; 2018.
14. Zegpi R A, He L, Yu Q, K. S. J, Santen V L v, Toro H. Limited protection conferred by recombinant Newcastle disease virus expressing infectious bronchitis spike protein. Avian Dis. 64:53-59; 2020.
15. Zhao W, Spatz S, Zhang Z, Wen G, Garcia M, Zsak L, Yu Q. Newcastle disease virus (NDV) recombinants expressing infectious laryngotracheitis virus (ILTV) glycoproteins gB and gD protect chickens against ILTV and NDV challenges. J Virol. 88:8397-8406; 2014.
16. Yu Q, Roth J P, Hu H, Estevez C N, Zhao W, Zsak L. Protection by Recombinant Newcastle Disease Viruses (NDV) Expressing the Glycoprotein (G) of Avian Metapneumovirus (aMPV) Subtype A or B against Challenge with Virulent NDV and aMPV. World J Vaccines. 3:130-139; 2013.
17. Chow Y H, Chiang B L, Lee Y L, Chi W K, Lin W C, Chen Y T, Tao M H. Development of Th1 and Th2 populations and the nature of immune responses to hepatitis B virus DNA vaccines can be modulated by codelivery of various cytokine genes. J Immunol. 160:1320-1329; 1998.
18. Du Y J, Jiang P, Li Y F, He H R, Jiang W M, Wang X L, Hong W B. Immune responses of two recombinant adenoviruses expressing VP1 antigens of FMDV fused with porcine granulocyte macrophage colony-stimulating factor. Vaccine. 25:8209-8219; 2007.
19. Hsieh M K, Wu C C, Lin T L. The effect of co-administration of DNA carrying chicken interferon-cgene on protection of chickens against infectious bursal disease by DNA-mediated vaccination. Vaccine. 24:6955-6965; 2006.
20. Heufler C, Koch F, Schuler G. Granulocyte/macrophage colony stimulating factor and interleukin 1 mediate the maturation of murine epidermal langerhans cells into potent immunostimulatory dendritic cells. J Exp Med. 167:700-705; 1988.
21. Foss D L, Bennaars A M, Pennell C A, Moody M D, Murtaugh M P. Differentiation of porcine dendritic cells by granulocyte macrophage colony-stimulating factor expressed in Pichia pastoris. Vet Immunol Immunopathol. 91:205-215; 2003.
22. Inaba K, Inaba M, Romani N, Aya H, Deguchi M, Ikehara S, Muramatsu S, Steinman R T. Generation of large numbers of dendritic cells from mouse bone marrow cultures supplemented with granulocyte/macrophage colony-stimulating factor. J Exp Med. 176:1693-1702; 1992.
23. Shi Y F, Liu C H, Roberts A I, Das J, Xu G W, Ren G W, Zhang Y Y, Zhang L Y, Yuan Z R, Tan H S W et al. Granulocyte-macrophage colony-stimulating factor (GM-CSF) and T-cell responses: what we do and don't know. Cell Res. 16:126-133; 2006.
24. Tazi A, Bouchonnet F, Grandsaigne M, Boumsell L, Hance I A J, Soler P. Evidence that granulocyte macrophage-colony stimulating factor regulates the distribution and differentiated state of dendritic cells/Langerhans cells in human lung and lung cancers. J Clin Invest. 91:566-576; 1993.
25. Andrew M, Morris K, Coupar B, Sproat K, Oke P, Bruce M, Broadway M, Morrissy C, Strom D. Porcine interleukin-3 enhances DNA vaccination against classical swine fever. Vaccine. 24:3241-3247; 2006.
26. Parker J N, Pfister L A, Quenelle D, Gillespie G Y, Markert J M, Kern E R, Whitley R J. Genetically engineered herpes simplex viruses that express IL-12 or GM-CSF as vaccine candidates. Vaccine. 24:1644-1652; 2006.
27. Tan B, Wang H, Shang L, Yang T. Coadministration of chicken GM-CSF with a DNA vaccine expressing infectious bronchitis virus (IBV) S1 glycoprotein enhances the specific immune response and protects against IBV infection. Arch Virol. 154:1117-1124; 2009.
28. Zeshan B, Mushtaq M H, Wang X, Li W, Jiang P. Protective immune responses induced by in ovo immunization with recombinant adenoviruses expressing spike (S1) glycoprotein of infectious bronchitis virus fused/co-administered with granulocyte-macrophage colony stimulating factor. Vet Microbiol. 148:8-17; 2011.
29. Toro H, van Santen V L, Li L, Lockaby S B, van Santen E, Hoerr F J. Epidemiological and experimental evidence for immunodeficiency affecting avian infectious bronchitis. Avian Pathol. 35:1-10; 2006.
30. Gallardo R A, Hoerr F J, Berry W D, van Santen V L, Toro H. Infectious bronchitis virus in testicles and venereal transmission. Avian Dis. 55:255-258; 2011.
31. Villegas P. Titration of biological suspensions. In: Dufour-Zavala L, Jackwood M W, Lee M D, Lupiani B, Reed W M, Spackman E, Woolcock P R, editors. A laboratory manual for the isolation, identification and characterization of avian pathogens. 6^{th} ed. Athens, G A: American Association of Avian Pathologists. p. 355-360; 2016.
32. Ghetas A M, van Santen V L, Joiner K, Toro H. Kidney cell-adapted infectious bronchitis ArkDPI vaccine confers effective protection against challenge. Avian Dis. 60:418-423; 2016.
33. He L, Zhang Z, Yu Q. Expression of two foreign genes by a Newcastle disease virus vector from the optimal insertion sites through a combination of the ITU and IRES-dependent expression approaches. Front Microbiol. 11:769; 2020.
34. Estevez C, King D, Seal B, Yu Q. Evaluation of Newcastle disease virus chimeras expressing the Hemagglutinin-Neuraminidase protein of velogenic strains in the context of a mesogenic recombinant virus backbone. Virus Res. 129:182-190; 2007.
35. Alexander D J, Senne D A. Newcastle disease virus and other avian paramyxoviruses. In: Dufour-Zavala L, Swayne D E, Glisson J R, Pearson J E, Reed W M, Jackwood M W, Woolcock P R, editors. A laboratory manual for the isolation, identification and characterization of avian pathogens. Fifth ed. Athens, G A: American Association of Avian Pathologists. p. 135-141; 2008.
36. Hu H, Roth J, Estevez C N, Zsak L, Liu B, Yu Q. Generation and evaluation of a recombinant Newcastle disease virus expressing the glycoprotein (G) of avian metapneumovirus subgroup C as a bivalent vaccine in turkeys. Vaccine. 29:8624-8633; 2011.
37. Wackenell P S, Thayer S G, Beard C W. Serologic procedures. In: Williams S M, editor. A laboratory manual for the isolation and identification, and characterization of avian pathogens. 6^{th} ed. Athens, G A: American Association of Avian Pathologists. p. 343-354; 2016.
38. Callison S A, Hilt D A, Boynton T O, Sample B F, Robison R, Swayne D E, Jackwood M W. Development and evaluation of a real-time Taqman rt-PCR assay for the detection of infectious bronchitis virus from infected chickens. J Virol Methods. 138:60-65; 2006.
39. Bertran K, Lee D H, Criado M F, Balzli C L, Killmaster L F, Kapczynski D R, Swayne D E. Maternal antibody inhibition of recombinant Newcastle disease virus vectored vaccine in a primary or booster avian influenza vaccination program of broiler chickens. Vaccine. 36:6361-6372; 2018.
40. Murr M, Grund C, Breithaupt A, Mettenleiter T C, Roemer-Oberdoerfer A. Protection of chickens with maternal immunity against avian influenza virus (AIV) by vaccination with a novel recombinant Newcastle disease virus vector. Avian Dis. 64:427-436; 2020.
41. Jackwood M W, Hilt D A, Lee C W, Kwon H M, Callison S A, Moore K M, Moscoso H, Sellers H, Thayer S. Data from 11 years of molecular typing infectious bronchitis virus field isolates. Avian Dis. 49:614-618; 2005.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of references are made herein. The cited references are incorporated by reference herein in their entireties. In the event that there is an inconsistency between a definition of a term in the specification as compared to a definition of the term in a cited reference, the term should be interpreted based on the definition in the specification.

1. A recombinant Newcastle disease virus vector comprising:
a nucleic acid encoding an infectious bronchitis virus (IBV) spike ectodomain (Se); and
a nucleic acid encoding a protein having granulocyte-macrophage colony-stimulating factor (GM-CSF) activity.

2. The vector of claim 1, wherein the protein having GM-CSF activity has an amino acid sequence having at least 90% sequence identity to SEQ ID NO: 121.

3. The vector of claim 1, wherein the Se is derived from Arkansas strain IBV.

4. The vector of claim 1, wherein the Se comprises a multimerization domain.

5. (canceled)

6. The vector of claim 4, wherein the amino acid sequence of the multimerization domain is SEQ ID NO: 122.

7. The vector of claim 1, wherein the recombinant Newcastle disease virus is LaSota strain of Newcastle disease virus.

8. The vector of claim 1, wherein the IBV spike ectodomain has the amino acid sequence SEQ ID NO: 120.

9. A polynucleotide comprising:
(i) a nucleic acid encoding recombinant Newcastle disease virus;
(ii) a nucleic acid encoding infectious bronchitis virus (IBV) spike ectodomain (Se); and
(iii) a nucleic acid encoding a protein having granulocyte-macrophage colony-stimulating factor (GM-CSF) activity;
wherein (i)-(iii) are operably linked to one or more promoters.

10. The polynucleotide of claim 9, wherein the protein having GM-CSF activity has an amino acid sequence having at least 90% sequence identity to SEQ ID NO: 121.

11. The polynucleotide of claim 9, wherein the Se is derived from Arkansas strain IBV.

12. The polynucleotide of claim 9, wherein the Se comprises a multimerization domain.

13. (canceled)

14. The polynucleotide of claim 12, wherein the multimerization domain is SEQ ID NO: 122.

15. The polynucleotide of claim 9, wherein the recombinant Newcastle disease virus is LaSota strain of Newcastle disease virus.

16. The polynucleotide of claim 9, wherein the amino acid sequence of the Se is SEQ ID NO: 120.

17. A pharmaceutical composition comprising the recombinant Newcastle disease virus vector of claim 1; and a pharmaceutically acceptable carrier.

18. A method of eliciting an immune response against infectious bronchitis virus (IBV), the method comprising:
administering an effective amount of the pharmaceutical composition of claim 17 to a subject to elicit an immune response against IBV.

19. (canceled)

20. The method of claim 18, further comprising administering a live-attenuated IBV vaccine to the subject.

21. The method of claim 20, wherein the live-attenuated IBV vaccine is a Mass strain live-attenuated vaccine.

22. (canceled)

23. The method of claim 18, wherein subjects administered the pharmaceutical composition exhibit greater protection against challenge by virulent IBV relative to subjects administered a composition not comprising a polynucleotide encoding GM-CSF.

24. A method of generating recombinant Newcastle disease virus vector comprising:
expressing the polynucleotide of claim 9 in a cell to generate a recombinant Newcastle disease virus vector.


SEQUENCE LISTING (124 sequences)

SEQ ID NO: 1 - protein, 1162 aa, Infectious Bronchitis Virus
MLVTPLLLVTLLCALCSAVLYDSSSYVYYYQSAFRPPSGWHLQGGAYAVVNISSEFNNAGSSSGCTVGIIHGGRVVNASSIAMTAPSSGMAWSSSQFCTAHCNFSDTTVFVTHCYKHGGCPLTGMLQQNLIRVSAMKNGQLFYNLTVSVAKYPTFRSFQCVNNLTSVYLNGDLVYTSNETIDVTSAGVYFKAGGPITYKVMREVKALAYFVNGTAQDVILCDGSPRGLLACQYNTGNFSDGFYPFTNSSLVKQKFIVYRENSVNTTCTLHNFIFHNETGANPNPSGVQNIQTYQTKTAQSGYYNFNFSFLSSFVYKESNFMYGSYHPSCKFRLETINNGLWFNSLSVSIAYGPLQGGCKQSVFKGRATCCYAYSYGGPSLCKGVYSGELDHNFECGLLVYVTKSGGSRIQTATEPPVITQNNYNNITLNTCVDYNIYGRTGQGFITNVTDSAVSYNYLADAGLAILDTSGSIDIFVVQGEYGLNYYKVNPCEDVNQQFVVSGGKLVGILTSRNETGSQLLENQFYIKITNGTRRFRRSITENVANCPYVSYGKFCIKPDGSIATIVPKQLEQFVAPLFNVTENVLIPNSFNLTVTDEYIQTRMDKVQINCLQYVCGSSLDCRKLFQQYGPVCDNILSVVNSVGQKEDMELLNFYSSTKPAGFNTPVLSNVSTGEFNISLLLTNPSSRRKRSLIEDLLFTSVESVGLPTNDAYKNCTAGPLGFFKDLACAREYNGLLVLPPIITAEMQALYTSSLVASMAFGGITAAGAIPFATQLQARINHLGITQSLLLKNQEKIAASFNKAIGHMQEGFRSTSLALQQIQDVVSKQSAILTETMASLNKNFGAISSVIQEIYQQFDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDVVTLTSCQANYVSVNKTVITTFVDNDDFDFNDELSKWWNDTKHELPDFDKFNYTVPILDIDSEIDRIQGVIQGLNDSLIDLEKLSILKTYIKWPWYVWLAIAFATIIFILILGWVFFMTGCCGCCCGCFGIMPLMSKCGKKSSYYTTFDNDVVTEQYRPKKSV

SEQ ID NO: 2 - protein, 17 aa, Infectious Bronchitis Virus
MLVTPLLLVTLLCALCS

SEQ ID NO: 3 - protein, 515 aa, Infectious Bronchitis Virus
AVLYDSSSYVYYYQSAFRPPSGWHLQGGAYAVVNISSEFNNAGSSSGCTVGIIHGGRVVNASSIAMTAPSSGMAWSSSQFCTAHCNFSDTTVFVTHCYKHGGCPLTGMLQQNLIRVSAMKNGQLFYNLTVSVAKYPTFRSFQCVNNLTSVYLNGDLVYTSNETIDVTSAGVYFKAGGPITYKVMREVKALAYFVNGTAQDVILCDGSPRGLLACQYNTGNFSDGFYPFTNSSLVKQKFIVYRENSVNTTCTLHNFIFHNETGANPNPSGVQNIQTYQTKTAQSGYYNFNFSFLSSFVYKESNFMYGSYHPSCKFRLETINNGLWFNSLSVSIAYGPLQGGCKQSVFKGRATCCYAYSYGGPSLCKGVYSGELDHNFECGLLVYVTKSGGSRIQTATEPPVITQNNYNNITLNTCVDYNIYGRTGQGFITNVTDSAVSYNYLADAGLAILDTSGSIDIFVVQGEYGLNYYKVNPCEDVNQQFVVSGGKLVGILTSRNETGSQLLENQFYIKITNGT

SEQ ID NO: 4 - protein, 5 aa, Infectious Bronchitits Virus
RRFRR

SEQ ID NO: 5 - protein, 554 aa, Infectious Bronchitits Virus
SITENVANCPYVSYGKFCIKPDGSIATIVPKQLEQFVAPLFNVTENVLIPNSFNLTVTDEYIQTRMDKVQINCLQYVCGSSLDCRKLFQQYGPVCDNILSVVNSVGQKEDMELLNFYSSTKPAGFNTPVLSNVSTGEFNISLLLTNPSSRRKRSLIEDLLFTSVESVGLPTNDAYKNCTAGPLGFFKDLACAREYNGLLVLPPIITAEMQALYTSSLVASMAFGGITAAGAIPFATQLQARINHLGITQSLLLKNQEKIAASFNKAIGHMQEGFRSTSLALQQIQDVVSKQSAILTETMASLNKNFGAISSVIQEIYQQFDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDVVTLTSCQANYVSVNKTVITTFVDNDDFDFNDELSKWWNDTKHELPDFDKFNYTVPILDIDSEIDRIQGVIQGLNDSLIDLEKLSILKTYIK

SEQ ID NO: 6 - protein, 67 aa, Infectious Bronchitis Virus
VWLAIAFATIIFILILGWVFFMTGCCGCCCGCFGIMPLMSKCGKKSSYYTTFDNDVVTEQYRPKKSV

SEQ ID NO: 7 - protein, 20 aa, Infectious Bronchitis Virus
VWLAIAFATIIFILILGWVF

SEQ ID NO: 8 - protein, 46 aa, Infectious Bronchitis Virus
MTGCCGCCCGCFGIMPLMSKCGKKSSYYTTFDNDVVTEQYRPKKSV

SEQ ID NO: 9 - protein, 1162 aa, Infectious Bronchitis Virus
MLVTPLLLVTLLCALCSAALYDSSSYVYYYQSAFRPPDGWHLHGGAYAVVNISSESNNAGSSSGCTVGIIHGGRVVNASSIAMTAPSSGMAWSSSQFCTAYCNFSDTTVFVTHCYKHVGCPITGMLQQHSIRVSAMKNGQLFYNLTVSVAKYPTFKSFQCVNNLTSVYLNGDLVYTSNETTDVTSAGVYFKAGGPITYKVMREVRALAYFVNGTAQDVILCDGSPRGLLACQYNTGNFSDGFYPFTNSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPLLCKGVYSGELDHNFECGLLVYVTKSGGSRIQTATEPPVITQHNYNNITLNTCVDYNIYGRTGQGFITNVTDSAVSYNYLADAGLAILDTSGSIDIFVVQSEYGLNYYKVNPCEDVNQQFVVSGGKLVGILTSRNETGSQLLENQFYIKITNGTRRFRRSITESVENCPYVSYGKFCIKPDGSIATIVPKQLEQFVAPLLNVTENVLIPNSFNLTVTDEYIQTRMDKVQINCLQYICGNSLECRNLFQQYGPVCDNILSVVNSVGQKEDMELLNFYSSTKPAGFNTPVLSNVSTGEFNISLFLTTPSSPRRRSFIEDLLFTSVESVGLPTDDAYKNCTAGPLGFLKDLVCAREYNGLLVLPPIITAEMQTLYTSSLVASMAFGGITAAGAIPFATQLQARINHLGITQSLLLKNQEKIAASFNKAIGHMQEGFRSTSLALQQIQDVVNKQSAILTETMASLNKNFGAISSVIQEIYQQLDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDIVTLTSCQVNYVSVNKTVITTFVDNDDFDFNDELSKWWNDTKHELPDFDKFNYTVPILDIDSEIDRIQGVIQGLNDSLIDLEKLSILKTYIKWPWYVWLAIAFATIIFILILGWVFFMTGCCGCCCGCFGIMPLMSKCGKKSSYYTTFDNDVVTEQYRPKKSV

SEQ ID NO: 10 - protein, 17 aa, Infectious Bronchitis Virus
MLVTPLLLVTLLCALCS

SEQ ID NO: 11 - protein, 515 aa, Infectious Bronchitis Virus
AALYDSSSYVYYYQSAFRPPDGWHLHGGAYAVVNISSESNNAGSSSGCTVGIIHGGRVVNASSIAMTAPSSGMAWSSSQFCTAYCNFSDTTVFVTHCYKHVGCPITGMLQQHSIRVSAMKNGQLFYNLTVSVAKYPTFKSFQCVNNLTSVYLNGDLVYTSNETTDVTSAGVYFKAGGPITYKVMREVRALAYFVNGTAQDVILCDGSPRGLLACQYNTGNFSDGFYPFTNSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPLLCKGVYSGELDHNFECGLLVYVTKSGGSRIQTATEPPVITQHNYNNITLNTCVDYNIYGRTGQGFITNVTDSAVSYNYLADAGLAILDTSGSIDIFVVQSEYGLNYYKVNPCEDVNQQFVVSGGKLVGILTSRNETGSQLLENQFYIKITNGT

SEQ ID NO: 12 - protein, 5 aa, Infectious Bronchitis Virus
RRFRR

SEQ ID NO: 13 - protein, 554 aa, Infectious Bronchitis Virus
SITESVENCPYVSYGKFCIKPDGSIATIVPKQLEQFVAPLLNVTENVLIPNSFNLTVTDEYIQTRMDKVQINCLQYICGNSLECRNLFQQYGPVCDNILSVVNSVGQKEDMELLNFYSSTKPAGFNTPVLSNVSTGEFNISLFLTTPSSPRRRSFIEDLLFTSVESVGLPTDDAYKNCTAGPLGFLKDLVCAREYNGLLVLPPIITAEMQTLYTSSLVASMAFGGITAAGAIPFATQLQARINHLGITQSLLLKNQEKIAASFNKAIGHMQEGFRSTSLALQQIQDVVNKQSAILTETMASLNKNFGAISSVIQEIYQQLDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDIVTLTSCQVNYVSVNKTVITTFVDNDDFDFNDELSKWWNDTKHELPDFDKFNYTVPILDIDSEIDRIQGVIQGLNDSLIDLEKLSILKTYIK

SEQ ID NO: 14 - protein, 67 aa, Infectious Bronchitis Virus; identical to SEQ ID NO: 6

SEQ ID NO: 15 - protein, 20 aa, Infectious Bronchitis Virus
VWLAIAFATIIFILILGWVF

SEQ ID NO: 16 - protein, 47 aa, Infectious Bronchitis Virus
FMTGCCGCCCGCFGIMPLMSKCGKKSSYYTTFDNDVVTEQYRPKKSV

SEQ ID NO: 17 - protein, 1165 aa, Infectious Bronchititis Virus
MLVKSLFLVTILCALCSANLFDSDNNYVYYYQSAFRPPNGWHLQGGAYAVVNSTNYTNNAGSAHQCTVGVIKDVYNQSVASIAMKAPLQGMAWSKSQFCSAHCNFSEITVFVTHCYSSGSGSCPITGMIPRDHIRISAMKNGSLFYNLTVSVSKYPNFKSFQCVNNFTSVYLNGDLVFTSNKTTDVTSAGVYFKAGGPVNYSIMKEFKVLAYFVNGTAQDVVLCDNSPKGLLACQYNTGNFSDGFYPFTNSTLVREKFIVYRESSVNTILALTNFTFTNVSNAQPNSGGVNTFHLYQTQTAQSGYYNFNLSFLSQFVYKASDFMYGSYHPSCSFRPETINSGLWFNSLSVSLTYGPLQGGCKQSVFSGKATCCYAYSYKGPMACKGVYSFQLSTNFECGLLVYVTKSDGSRIQTRTEPLVLTQYNYNNITLDKCVAYNIYGRVGQGFITNVTDSAANFSYLADGGLAILDTSGAIDVFVVQGIYGLNYYKVNPCEDVNQQFVVSGGNIVGILTSRNETGSEQVENQFYVKLTNSSHRRRRSIGQNVTSCPYVSYGRFCIEPDGSLKMIVPEELKQFVAPLLNITESVLIPNSFNLTVTDEYIQTRMDKVQINCLQYVCGNSLECRKLFQQYGPVCDNILSVVNSVSQKEDMELLSFYSSTKPKGYDTPVLSNVSTGEFNISLLLKPPSSPSGRSFIEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGHMQEGFRSTSLALQQIQDVVNKQSAILTETMNSLNKNFGAITSVIQDIYAQLDAIQADAQVDRLITGRLSTLSVLAFAKQSEYIRVSQQRELATQKINECVKSQSNRYGFCGSGRHVLSIPQNAPNGIVFIHFTYTPESFVNVTAIVGFCVNPANASQYAIVPANGRGIFIQVNGTYYITARDMYMPRDITAGDIVTLTSCQANYVNVNKTVITTFVEDDDFDFDDELSKWWNDTKHQLPDFDDFNYTVPILNISGEIDYIQGVIQGLNDSLINLEELSIIKTYIKWPWYVWLAIGFAIIIFILILGWVFFMTGCCGCCCGCFGIIPLMSKCGKKSSYYTTFDNDVVTEQYRPKKSV

SEQ ID NO: 18 - protein, 17 aa, Infectious Bronchitis Virus
MLVKSLFLVTILCALCS

SEQ ID NO: 19 - protein, 519 aa, Infectious Bronchitis Virus
ANLFDSDNNYVYYYQSAFRPPNGWHLQGGAYAVVNSTNYTNNAGSAHQCTVGVIKDVYNQSVASIAMKAPLQGMAWSKSQFCSAHCNFSEITVFVTHCYSSGSGSCPITGMIPRDHIRISAMKNGSLFYNLTVSVSKYPNFKSFQCVNNFTSVYLNGDLVFTSNKTTDVTSAGVYFKAGGPVNYSIMKEFKVLAYFVNGTAQDVVLCDNSPKGLLACQYNTGNFSDGFYPFTNSTLVREKFIVYRESSVNTILALTNFTFTNVSNAQPNSGGVNTFHLYQTQTAQSGYYNFNLSFLSQFVYKASDFMYGSYHPSCSFRPETINSGLWFNSLSVSLTYGPLQGGCKQSVFSGKATCCYAYSYKGPMACKGVYSFQLSTNFECGLLVYVTKSDGSRIQTRTEPLVLTQYNYNNITLDKCVAYNIYGRVGQGFITNVTDSAANFSYLADGGLAILDTSGAIDVFVVQGIYGLNYYKVNPCEDVNQQFVVSGGNIVGILTSRNETGSEQVENQFYVKLTNSSH

SEQ ID NO: 20 - protein, 5 aa, Infectious Bronchitis Virus
HRRRR

SEQ ID NO: 21 - protein, 554 aa, Infectious Bronchitis Virus
SIGQNVTSCPYVSYGRFCIEPDGSLKMIVPEELKQFVAPLLNITESVLIPNSFNLTVTDEYIQTRMDKVQINCLQYVCGNSLECRKLFQQYGPVCDNILSVVNSVSQKEDMELLSFYSSTKPKGYDTPVLSNVSTGEFNISLLLKPPSSPSGRSFIEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGHMQEGFRSTSLALQQIQDVVNKQSAILTETMNSLNKNFGAITSVIQDIYAQLDAIQADAQVDRLITGRLSTLSVLAFAKQSEYIRVSQQRELATQKINECVKSQSNRYGFCGSGRHVLSIPQNAPNGIVFIHFTYTPESFVNVTAIVGFCVNPANASQYAIVPANGRGIFIQVNGTYYITARDMYMPRDITAGDIVTLTSCQANYVNVNKTVITTFVEDDDFDFDDELSKWWNDTKHQLPDFDDFNYTVPILNISGEIDYIQGVIQGLNDSLINLEELSIIKTYIK

SEQ ID NO: 22 - protein, 67 aa, Infectious Bronchitis Virus
VWLAIGFAIIIFILILGWVFFMTGCCGCCCGCFGIIPLMSKCGKKSSYYTTFDNDVVTEQYRPKKSV

SEQ ID NO: 23 - protein, 20 aa, Infectious Bronchitis Virus
VWLAIGFAIIIFILILGWVF

SEQ ID NO: 24 - protein, 47 aa, Infectious Bronchitis Virus
FMTGCCGCCCGCFGIIPLMSKCGKKSSYYTTFDNDVVTEQYRPKKSV

SEQ ID NO: 25 - protein, 1165 aa, Infectious Bronchitis Virus
MLVKSLFLVTILCALCSANLFDSDNNYVYYYQSAFRPPNGWHLQGGAYAVVNSTNYTNNAGSAHECTVGVIKDVYNQSVASIAMTAPLQGMAWSKSQFCSAHCNFSEITVFVTHCYSSGSGSCPITGMIPRDHIRISAMKNGSLFYNLTVSVSKYPNFKSFQCVNNFTSVYLNGDLVFTSNKTTDVTSAGVYFKAGGPVNYSIMKEFKVLAYFVNGTAQDVVLCDNSPKGLLACQYNTGNFSDGFYPFTNSTLVREKFIVYRESSVNTTLALTNFTFTNVSNAQPNSGGVNTFHLYQTQTAQSGYYNFNLSFLSQFVYKASDFMYGSYHPSCSFRPETINSGLWFNSLSVSLTYGPLQGGCKQSVFSGKATCCYAYSYKGPMACKGVYSGELSTNFECGLLVYVTKSDGSRIQTRTEPLVLTQYNYNNITLDKCVAYNIYGRVGQGFITNVTDSAANFSYLADGGLAILDTSGAIDVFVVQGIYGLNYYKVNPCEDVNQQFVVSGGNIVGILTSRNETGSEQVENQFYVKLTNSSHRRRRSIGQNVTSCPYVSYGRFCIEPDGSLKMIVPEELKQFVAPLLNITESVLIPNSFNLTVTDEYIQTRMDKVQINCLQYVCGNSLECRKLFQQYGPVCDNILSVVNSVSQKEDMELLSFYSSTKPKGYDTPVLSNVSTGEFNISLLLKPPSSPSGRSFIEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGHMQEGFRSTSLALQQIQDVVNKQSAILTETMNSLNKNFGAITSVIQDIYAQLDAIQADAQVDRLITGRLSSLSVLASAKQSEYIRVSQQRELATQKINECVKSQSNRYGFCGSGRHVLSIPQNAPNGIVFIHFTYTPESFVNVTAIVGFCVNPANASQYAIVPANGRGIFIQVNGTYYITARDMYMPRDITAGDIVTLTSCQANYVNVNKTVITTFVEDDDFDFDDELSKWWNDTKHQLPDFDDFNYTVPILNISGEIDYIQGVIQGLNDSLINLEELSIIKTYIKWPWYVWLAIGFAIIIFILILGWVFFMTGCCGCCCGCFGIIPLMSKCGKKSSYYTTFDNDVVTEQYRPKKSV

SEQ ID NO: 26 - protein, 17 aa, Infectious Bronchitis Virus
MLVKSLFLVTILCALCS

SEQ ID NO: 27 - protein, 518 aa, Infectious Bronchitis Virus
ANLFDSDNNYVYYYQSAFRPPNGWHLQGGAYAVVNSTNYTNNAGSAHECTVGVIKDVYNQSVASIAMTAPLQGMAWSKSQFCSAHCNFSEITVFVTHCYSSGSGSCPITGMIPRDHIRISAMKNGSLFYNLTVSVSKYPNFKSFQCVNNFTSVYLNGDLVFTSNKTTDVTSAGVYFKAGGPVNYSIMKEFKVLAYFVNGTAQDVVLCDNSPKGLLACQYNTGNFSDGFYPFTNSTLVREKFIVYRESSVNTTLALTNFTFTNVSNAQPNSGGVNTFHLYQTQTAQSGYYNFNLSFLSQFVYKASDFMYGSYHPSCSFRPETINSGLWFNSLSVSLTYGPLQGGCKQSVFSGKATCCYAYSYKGPMACKGVYSGELSTNFECGLLVYVTKSDGSRIQTRTEPLVLTQYNYNNITLDKCVAYNIYGRVGQGFITNVTDSAANFSYLADGGLAILDTSGAIDVFVVQGIYGLNYYKVNPCEDVNQQFVVSGGNIVGILTSRNETGSEQVENQFYVKLTNSS

SEQ ID NO: 28 - protein, 5 aa, Infectious Bronchitis Virus
HRRRR

SEQ ID NO: 29 - protein, 554 aa, Infectious Bronchitis Virus
SIGQNVTSCPYVSYGRFCIEPDGSLKMIVPEELKQFVAPLLNITESVLIPNSFNLTVTDEYIQTRMDKVQINCLQYVCGNSLECRKLFQQYGPVCDNILSVVNSVSQKEDMELLSFYSSTKPKGYDTPVLSNVSTGEFNISLLLKPPSSPSGRSFIEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGHMQEGFRSTSLALQQIQDVVNKQSAILTETMNSLNKNFGAITSVIQDIYAQLDAIQADAQVDRLITGRLSSLSVLASAKQSEYIRVSQQRELATQKINECVKSQSNRYGFCGSGRHVLSIPQNAPNGIVFIHFTYTPESFVNVTAIVGFCVNPANASQYAIVPANGRGIFIQVNGTYYITARDMYMPRDITAGDIVTLTSCQANYVNVNKTVITTFVEDDDFDFDDELSKWWNDTKHQLPDFDDFNYTVPILNISGEIDYIQGVIQGLNDSLINLEELSIIKTYIK

SEQ ID NO: 30 - protein, 67 aa, Infectious Bronchitis Virus; identical to SEQ ID NO: 22

SEQ ID NO: 31 - protein, 20 aa, Infectious Bronchitis Virus
VWLAIGFAIIIFILILGWVF

SEQ ID NO: 32 - protein, 47 aa, Infectious Bronchitis Virus; identical to SEQ ID NO: 24

SEQ ID NO: 33 - protein, 1165 aa, Infectious Bronchitis Virus
MLVKPLFIVTALLSLCSGNLFDSNNTYVYYYQSAFRPFDGWHVQGGAYAVSSVFNRSNNANSHTCSVGTLGPSYNYSGAAVAMTAPHSGMNWSKDQFCTAHCNFSDIFVYVTHCYANNCVLVNNIGQGNIYIGAKRNGESIFNKILSVTSYPKFMSLHCVNNATSVYLNGNLVFTSNQTASVTGAGVYFKAGGPITYKVMKDFPVLAYFKNGTVHDVILCDSTPKGLLACQYNTGNFTDGLYPFTNVTESKEKFIVYGESSVTTLLTLTNYTFYNVSNAPPAGAYVENFVKYQTQTAQEGFYNFNFTFLQDSRFQYVPSDYDRGSYHKACDFRLESINNNLWFNSLSVSLTYGPIQGGCKQSVFSYKTTCCYAYSYNGPIECSGVYNGYRTHKYECGLLVYVSMTKGSRIQTSNVVPTVTQYNYNNITLDTCVDYSIYGKVGQGFITNVTDQLSSNNYLDQGGLAILDTSGAIDIFVVNGSSGRNYYKINPCSDVNQQFVVSGGNIVGILTLLNKTGSQQVEDQFFVKLVSGSHRKKRSVSTLPTVKNCPYTAYGKFCFNPDGEFYEITPAEIETYSSPLLNVSGFVLIPDTFNLTVTDEYIQTFMEKIQINCIQYVCGNSIECKKLFQQYGPVCDNILNIVYSLAQQDNMEFLQFYSATKPKGFDTIIFNNVSTDGFNLSLVLRQPSSPRGRSFIEDLLFTKVESLGLPGDTAYQKCTAGPLGFVKDLVCAQHYNGLLVLPPIITAEMQTMYTASLVASMAFGGITSAGAIPFATQIQARINHLGITQTVLQKNQEKIAASFNKAMAHMQDGFRATSLALQQIQDVLNRQGSVLQDAMNSLNKNFGAISSVIQDIYSRLDTLEANTQVDRLISGRLSSLSVLASAKQAEYAKVSQQRELAKQKINECVKSQSSRLGFCGAGRHVLTIPQNAPNGILLLHFTYTPETFKNVTAVVGFCVKPENGSEYAIVPANGRGIFIQVNGTYYITARDMYMPRSITAGDIVTLTSCQANYVNVNKTVITTFVDEDDFDFNDELSKWWNETKHEFPDFDQFNYTIPVLNITYDIDKIEEVIKGLNDSLIDLETLSILKTYIKWPWYVWLAIAFATIIFILILGWVFFMTGCCGCYCGCFGIIPLMSKCSKKSSYYTTFDNDVVTEQYRPKKSV

SEQ ID NO: 34 - protein, 17 aa, Infectious Bronchitis Virus
MLVKPLFIVTALLSLCS

SEQ ID NO: 35 - protein, 516 aa, Infectious Bronchitis Virus
GNLFDSNNTYVYYYQSAFRPFDGWHVQGGAYAVSSVFNRSNNANSHTCSVGTLGPSYNYSGAAVAMTAPHSGMNWSKDQFCTAHCNFSDIFVYVTHCYANNCVLVNNIGQGNIYIGAKRNGESIFNKILSVTSYPKFMSLHCVNNATSVYLNGNLVFTSNQTASVTGAGVYFKAGGPITYKVMKDFPVLAYFKNGTVHDVILCDSTPKGLLACQYNTGNFTDGLYPFTNVTESKEKFIVYGESSVTTLLTLTNYTFYNVSNAPPAGAYVENFVKYQTQTAQEGFYNFNFTFLQDSRFQYVPSDYDRGSYHKACDFRLESINNNLWFNSLSVSLTYGPIQGGCKQSVFSYKTTCCYAYSYNGPIECSGVYNGYRTHKYECGLLVYVSMTKGSRIQTSNVVPTVTQYNYNNITLDTCVDYSIYGKVGQGFITNVTDQLSSNNYLDQGGLAILDTSGAIDIFVVNGSSGRNYYKINPCSDVNQQFVVSGGNIVGILTLLNKTGSQQVEDQFFVKLVSGS

SEQ ID NO: 36 - protein, 5 aa, Infectious Bronchitis Virus
HRKKR

SEQ ID NO: 37 - protein, 556 aa, Infectious Brochitis Virus
SVSTLPTVKNCPYTAYGKFCFNPDGEFYEITPAEIETYSSPLLNVSGFVLIPDTFNLTVTDEYIQTFMEKIQINCIQYVCGNSIECKKLFQQYGPVCDNILNIVYSLAQQDNMEFLQFYSATKPKGFDTIIFNNVSTDGFNLSLVLRQPSSPRGRSFIEDLLFTKVESLGLPGDTAYQKCTAGPLGFVKDLVCAQHYNGLLVLPPIITAEMQTMYTASLVASMAFGGITSAGAIPFATQIQARINHLGITQTVLQKNQEKIAASFNKAMAHMQDGFRATSLALQQIQDVLNRQGSVLQDAMNSLNKNFGAISSVIQDIYSRLDTLEANTQVDRLISGRLSSLSVLASAKQAEYAKVSQQRELAKQKINECVKSQSSRLGFCGAGRHVLTIPQNAPNGILLLHFTYTPETFKNVTAVVGFCVKPENGSEYAIVPANGRGIFIQVNGTYYITARDMYMPRSITAGDIVTLTSCQANYVNVNKTVITTFVDEDDFDFNDELSKWWNETKHEFPDFDQFNYTIPVLNITYDIDKIEEVIKGLNDSLIDLETLSILKTYIK

SEQ ID NO: 38 - protein, 67 aa, Infectious Bronchitis Virus
VWLAIAFATIIFILILGWVFFMTGCCGCYCGCFGIIPLMSKCSKKSSYYTTFDNDVVTEQYRPKKSV

SEQ ID NO: 39 - protein, 20 aa, Infectious Bronchitis Virus
VWLAIAFATIIFILILGWVF

SEQ ID NO: 40 - protein, 47 aa, Infectious Bronchitis Virus
FMTGCCGCYCGCFGIIPLMSKCSKKSSYYTTFDNDVVTEQYRPKKSV

SEQ ID NO: 41 - protein, 1162 aa, Infectious Bronchitis Virus
MLVTPLLLVTLLCALCSAVLYDSSSYVYYYQSAFRPPSGWHLQGGAYAVVNISSEFNNAGSSSGCTVGIIHGGRVVNASSIAMTAPSSGMAWSSSQFCTAHCNFSDTTVFVTHCYKHGGCPITGMLQQNFIRVSAMKNGQLFYNLTVSVAKYPTFRSFQCVNNLTSVYLNGDLVYTSNETIDVTSAGVYFKAGGPITYKVMREVKALAYFVNGTAQDVILCDGSPRGLLACQYNTGNFSDGFYPFTNSSLVKQKFIVYRENSVNTTCTLHNFIFHNETGANPNPSGVQNIQTYQTKTAQSGYYNFNFSFLSSFVYKESNFMYGSYHPSCKFRLETINNGLWFNSLSVSIAYGPLQGGCKQSVFRGRATCCYAYSYGGPSLCKGVYSGELDHNFECGLLVYVTKSDGSRIQTATEPPVITQHNYNNITLNTCVDYNIYGRTGQGFITNVTDSAVSYNYLADAGLAILDTSGSIDIFVVQGEYGLNYYKVNPCEDVNQQFVVSGGKLVGILTSRNETGSQLLENQFYIKITNGTRRFRRSITENVANCPYVSYGKFCIKPDGSIATIVPKQLEQFVAPLFNVTENVLIPNSFNLTVTDEYIQTRMDKVQINCLQYVCGSSLDCRNLFQQYGPVCDNILSVVNSVGQKEDMELLNFYSSTKPAGFNTPVLSNVSTGEFNISLLLTTPSSRRRRSVIEDLLFTSVESVGLPTDDAYKNCTAGPLGFLKDLACAREYNGLLVLPPIITAEMQALYTSSLVASMAFGGITAAGAIPFATQLQARINHLGITQSLLLKNQEKIAASFNKAIGHMQEGFRSTSLALQQIQDVVSKQSAILTETMASLNKNFGAISSVIQEIYQQFDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDVVTLTSCQANYVIVNKTVITTFVDNDDFDFNDELSKWWNDTKHELPDFDKFNYTVPILDIDSEIDRIQGVIQGLNDSLIDLEKLSILKTYIKWPWYVWLAIAFATIIFILILGWVFFMTGCCGCCCGCFGIMPLMSKCGKKSSYYTTFDNDVVTEQYRPKKSV

SEQ ID NO: 42 - protein, 17 aa, Infectious Bronchitis Virus
MLVTPLLLVTLLCALCS

SEQ ID NO: 43 - protein, 515 aa, Infectious Bronchitis Virus
AVLYDSSSYVYYYQSAFRPPSGWHLQGGAYAVVNISSEFNNAGSSSGCTVGIIHGGRVVNASSIAMTAPSSGMAWSSSQFCTAHCNFSDTTVFVTHCYKHGGCPITGMLQQNFIRVSAMKNGQLFYNLTVSVAKYPTFRSFQCVNNLTSVYLNGDLVYTSNETIDVTSAGVYFKAGGPITYKVMREVKALAYFVNGTAQDVILCDGSPRGLLACQYNTGNFSDGFYPFTNSSLVKQKFIVYRENSVNTTCTLHNFIFHNETGANPNPSGVQNIQTYQTKTAQSGYYNFNFSFLSSFVYKESNFMYGSYHPSCKFRLETINNGLWFNSLSVSIAYGPLQGGCKQSVFRGRATCCYAYSYGGPSLCKGVYSGELDHNFECGLLVYVTKSDGSRIQTATEPPVITQHNYNNITLNTCVDYNIYGRTGQGFITNVTDSAVSYNYLADAGLAILDTSGSIDIFVVQGEYGLNYYKVNPCEDVNQQFVVSGGKLVGILTSRNETGSQLLENQFYIKITNGT

SEQ ID NO: 44 - protein, 5 aa, Infectious Bronchitis Virus
RRFRR

SEQ ID NO: 45 - protein, 554 aa, Infectious Bronchitis Virus
SITENVANCPYVSYGKFCIKPDGSIATIVPKQLEQFVAPLFNVTENVLIPNSFNLTVTDEYIQTRMDKVQINCLQYVCGSSLDCRNLFQQYGPVCDNILSVVNSVGQKEDMELLNFYSSTKPAGFNTPVLSNVSTGEFNISLLLTTPSSRRRRSVIEDLLFTSVESVGLPTDDAYKNCTAGPLGFLKDLACAREYNGLLVLPPIITAEMQALYTSSLVASMAFGGITAAGAIPFATQLQARINHLGITQSLLLKNQEKIAASFNKAIGHMQEGFRSTSLALQQIQDVVSKQSAILTETMASLNKNFGAISSVIQEIYQQFDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDVVTLTSCQANYVIVNKTVITTFVDNDDFDFNDELSKWWNDTKHELPDFDKFNYTVPILDIDSEIDRIQGVIQGLNDSLIDLEKLSILKTYIK

SEQ ID NO: 46 - protein, 67 aa, Infectious Bronchitis Virus; identical to SEQ ID NO: 6

SEQ ID NO: 47 - protein, 20 aa, Infectious Bronchitis Virus
VWLAIAFATIIFILILGWVF

SEQ ID NO: 48 - protein, 47 aa, Infectious Bronchitis Virus; identical to SEQ ID NO: 16

SEQ ID NO: 49 - protein, 1162 aa, Infectious Bronchitis Virus
MLVTPLLLVTLLCALCSAVLYDSSSYVYYYQSAFRPPNGWHLHGGAYAVVNISSESNNAGSSPGCIVGTIHGGRVVNASSIAMTAPSSGMAWSSSQFCTAHCNFSDTTVFVTHCYKYDGCPITGMLQKNFIRVSAMKNGQLFYNLTVSVAKYPTFKSFQCVNNLTSVYLNGDLVYTSNETTDVTSAGVYFKAGGPITYKVMREVKALAYFVNGTAQDVILCDGSPRGLLACQYNTGNFSDGFYPFINSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPSLCKGVYSGELDLNFECGLLVYVTKSGGSRIQTATEPPVITQHNYNNITLNTCVDYNIYGRTGQGFITNVTDSAVSYNYLADAGLAILDTSGSIDIFVVQGEYGLNYYKVNPCEDVNQQFVVSGGKLVGILTSRNETGSQLLENQFYIKITNGTRRFRRSITENVANCPYVSYGKFCIKPDGSIATIVPKQLEQFVAPLFNVTENVLIPNSFNLTVTDEYIQTRMDKVQINCLQYVCGSSLDCRKLFQQYGPVCDNILSVVNSVGQKEDMELLNFYSSTKPAGFNTPVLSNVSTGEFNISLLLTTPSSRRRRSFIEDLLFTSVESVGLPTDDAYKNCTAGPLGFLKDLACAREYNGLLVLPPIITAEMQTLYTSSLVASMAFGGITAAGAIPFATQLQARINHLGITQSLLLKNQEKIAASFNKAIGHMQEGFRSTSLALQQIQDVVNKQSAILTETMASLNKNFGAISSVIQEIYQQLDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPSNGRGIFIQVNGSYYITARDMYMPRAITAGDVVTLTSCQANYVSVNKTVITTFVDNDDFDFNDELSKWWNDTKHELPDFDKFNYTVPILDIDSEIDRIQGVIQGLNDSLIDLEKLSILKTYIKWPWYVWLAIAFATIIFILILGWVFFMTGCCGCCCGCFGIMPLMSKCGKKSSYYTTFDNDVVTEQYRPKKSV

SEQ ID NO: 50 - protein, 17 aa, Infectious Bronchitis Virus
MLVTPLLLVTLLCALCS

SEQ ID NO: 51 - protein, 515 aa, Infectious Bronchitis Virus
AVLYDSSSYVYYYQSAFRPPNGWHLHGGAYAVVNISSESNNAGSSPGCIVGTIHGGRVVNASSIAMTAPSSGMAWSSSQFCTAHCNFSDTTVFVTHCYKYDGCPITGMLQKNFIRVSAMKNGQLFYNLTVSVAKYPTFKSFQCVNNLTSVYLNGDLVYTSNETTDVTSAGVYFKAGGPITYKVMREVKALAYFVNGTAQDVILCDGSPRGLLACQYNTGNFSDGFYPFINSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPSLCKGVYSGELDLNFECGLLVYVTKSGGSRIQTATEPPVITQHNYNNITLNTCVDYNIYGRTGQGFITNVTDSAVSYNYLADAGLAILDTSGSIDIFVVQGEYGLNYYKVNPCEDVNQQFVVSGGKLVGILTSRNETGSQLLENQFYIKITNGT

SEQ ID NO: 52 - protein, 5 aa, Infectious Bronchitis Virus
RRFRR

SEQ ID NO: 53 - protein, 554 aa, Infectious Bronchitis Virus
SITENVANCPYVSYGKFCIKPDGSIATIVPKQLEQFVAPLFNVTENVLIPNSFNLTVTDEYIQTRMDKVQINCLQYVCGSSLDCRKLFQQYGPVCDNILSVVNSVGQKEDMELLNFYSSTKPAGFNTPVLSNVSTGEFNISLLLTTPSSRRRRSFIEDLLFTSVESVGLPTDDAYKNCTAGPLGFLKDLACAREYNGLLVLPPIITAEMQTLYTSSLVASMAFGGITAAGAIPFATQLQARINHLGITQSLLLKNQEKIAASFNKAIGHMQEGFRSTSLALQQIQDVVNKQSAILTETMASLNKNFGAISSVIQEIYQQLDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPSNGRGIFIQVNGSYYITARDMYMPRAITAGDVVTLTSCQANYVSVNKTVITTFVDNDDFDFNDELSKWWNDTKHELPDFDKFNYTVPILDIDSEIDRIQGVIQGLNDSLIDLEKLSILKTYIK

SEQ ID NO: 54 - protein, 67 aa, Infectious Bronchitis Virus; identical to SEQ ID NO: 6

SEQ ID NO: 55 - protein, 20 aa, Infectious Bronchitis Virus
VWLAIAFATIIFILILGWVF

SEQ ID NO: 56 - protein, 47 aa, Infectious Bronchitis Virus; identical to SEQ ID NO: 16

SEQ ID NO: 57 - protein, 1162 aa, Infectious Bronchitis Virus; identical to SEQ ID NO: 41

SEQ ID NO: 58 - protein, 17 aa, Infectious Bronchitis Virus
MLVTPLLLVTLLCALCS

SEQ ID NO: 59 - protein, 515 aa, Infectious Bronchitis Virus; identical to SEQ ID NO: 43

SEQ ID NO: 60 - protein, 5 aa, Infectious Bronchitis Virus
RRFRR

SEQ ID NO: 61 - protein, 554 aa, Infectious Bronchitis Virus; identical to SEQ ID NO: 45

SEQ ID NO: 62 - protein, 67 aa, Infectious Bronchitis Virus; identical to SEQ ID NO: 6

SEQ ID NO: 63 - protein, 20 aa, Infectious Bronchitis Virus
VWLAIAFATIIFILILGWVF

SEQ ID NO: 64 - protein, 47 aa, Infectious Bronchitis Virus; identical to SEQ ID NO: 16

SEQ ID NO: 65 - protein, 1162 aa, Infectious Bronchitis Virus
MLVTPLLLVTLLCALCSAVLYDSSSYVYYYQSAFRPPNGWHLHGGAYAVVNISSESNNAGSSPGCIVGTIHGGRVVNASSIAMTAPSSGMAWSSSQFCTAHCNFSDTTVFVTHCYKYDGCPITGMLQKNFIRVSAMKNGQLFYNLTVSVAKYPTFKSFQCVNNLTSVYLNGDLVYTSNETTDVTSAGVYFKAGGPITYKVMREVKALAYFVNGTAQDVILCDGSPRGLLACQYNTGNFSDGFYPFINSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPSLCKGVYSGELDLNFECGLLVYVTKSGGSRIQTATEPPVITQHNYNNITLNTCVDYNIYGRTGQGFITNVTDSAVSYNYLADAGLAILDTSGSIDIFVVQGEYGLNYYKVNPCEDVNQQFVVSGGKLVGILTSRNETGSQLLENQFYIKITNGTRRFRRSITENVANCPYVSYGKFCIKPDGSIATIVPKQLEQFVAPLFNVTENVLIPNSFNLTVTDEYIQTRMDKVQINCLQYVCGSSLDCRKLFQQYGPVCDNILSVVNSVGQKEDMELLNFYSSTKPAGFNTPVLSNVSTGEFNISLLLTTPSSRRRRSFIEDLLFTSVESVGLPTDDAYKNCTAGPLGFLKDLACAREYNGLLVLPPIITAEMQALYTSSLVASMAFGGITAAGAMPFATQLQARINHLGITQSLLLKNQEKIAASFNKAIGHMQEGFRSTSLALQQIQDVVSKQSAILTETMASLNKNFGAISSVIQEIYQQFDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDVVTLTSCQANYVIVNKTVITTFVDNDDFDFNDELSKWWNDTKHELPDFDKFNYTVPILDIDSEIDRIQGVIQGLNDSLIDLEKLSILKTYIKWPWYVWLAIAFATIIFILILGWVFFMTGCCGCCCGCFGIMPLMSKCGKKSSYYTTFDNDVVTEQYRPKKSV

SEQ ID NO: 66 - protein, 17 aa, Infectious Bronchitis Virus
MLVTPLLLVTLLCALCS

SEQ ID NO: 67 - protein, 515 aa, Infectious Bronchitis Virus; identical to SEQ ID NO: 51

SEQ ID NO: 68 - protein, 5 aa, Infectious Bronchitis Virus
RRFRR

SEQ ID NO: 69 - protein, 554 aa, Infectious Bronchitis Virus
SITENVANCPYVSYGKFCIKPDGSIATIVPKQLEQFVAPLFNVTENVLIPNSFNLTVTDEYIQTRMDKVQINCLQYVCGSSLDCRKLFQQYGPVCDNILSVVNSVGQKEDMELLNFYSSTKPAGFNTPVLSNVSTGEFNISLLLTTPSSRRRRSFIEDLLFTSVESVGLPTDDAYKNCTAGPLGFLKDLACAREYNGLLVLPPIITAEMQALYTSSLVASMAFGGITAAGAMPFATQLQARINHLGITQSLLLKNQEKIAASFNKAIGHMQEGFRSTSLALQQIQDVVSKQSAILTETMASLNKNFGAISSVIQEIYQQFDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDVVTLTSCQANYVIVNKTVITTFVDNDDFDFNDELSKWWNDTKHELPDFDKFNYTVPILDIDSEIDRIQGVIQGLNDSLIDLEKLSILKTYIK

SEQ ID NO: 70 - protein, 67 aa, Infectious Bronchitis Virus; identical to SEQ ID NO: 6

SEQ ID NO: 71 - protein, 20 aa, Infectious Bronchitis Virus
VWLAIAFATIIFILILGWVF

SEQ ID NO: 72 - protein, 47 aa, Infectious Bronchitis Virus; identical to SEQ ID NO: 16

SEQ ID NO: 73 - protein, 1162 aa, Infectious Bronchitis Virus
MLVTPLLLVTLLCALCSAVLYDSSSYVYYYQSAFRPPNGWHLHGGAYAVVNISSESNNAGSSPGCIVGTIHGGRVVNASSIAMTAPSSGMAWSSSQFCTAHCNFSDTTVFVTHCYKYDGCPITGMLQKNFIRVSAMKNGQLFYNLTVSVAKYPTFKSFQCVNNLTSVYLNGDLVYTSNETTDVTSAGVYFKAGGPITYKVMREVKALAYFVNGTAQDVILCDGSPRGLLACQYNTGNFSDGFYPFINSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPSLCKGVYSGELDLNFECGLLVYVTKSGGSRIQTATEPPVITQHNYNNITLNTCVDYNIYGRTGQGFITNVTDSAVSYNYLADAGLAILDTSGSIDIFVVQGEYGLNYYKVNPCEDVNQQFVVSGGKLVGILTSRNETGSQLLENQFYIKITNGTRRFRRSITENVANCPYVSYGKFCIKPDGSIATIVPKQLEQFVAPLFNVTENVLIPNSFNLTVTDEYIQTRMDKVQINCLQYVCGSSLDCRKLFQQYGPVCDNILSVVNSVGQKEDMELLNFYSSTKPAGFNTPVLSNVSTGEFNISLLLTTPSSRRRRSFIEDLLFTSVESVGLPTDDAYKNCTAGPLGFLKDLACAREYNGLLVLPPIITAEMQALYTSSLVASMAFGGITAAGAIPFATQLQARINHLGITQSLLLKNQEKIAASFNKAIGHMQEGFRSTSLALQQIQDVVSKQSAILTETMASLNKNFGAISSVIQEIYQQFDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDVVTLTSCQANYVIVNKTVITTFVDNDDFDFNDELSKWWNDTKHELPDFDKFNYTVPILDIDSEIDRIQGVIQGLNDSLIDLEKLSILKTYIKWPWYVWLAIAFATIIFILILGWVFFMTGCCGCCCGCFGIMPLMSKCGKKSSYYTTFDNDVVTEQYRPKKSV

SEQ ID NO: 74 - protein, 17 aa, Infectious Bronchitis Virus
MLVTPLLLVTLLCALCS

SEQ ID NO: 75 - protein, 515 aa, Infectious Bronchitis Virus; identical to SEQ ID NO: 51

SEQ ID NO: 76 - protein, 5 aa, Infectious Bronchitis Virus
FFRFF

SEQ ID NO: 77 - protein, 554 aa, Infectious Bronchitis Virus
SITENVANCPYVSYGKFCIKPDGSIATIVPKQLEQFVAPLFNVTENVLIPNSFNLTVTDEYIQTRMDKVQINCLQYVCGSSLDCRKLFQQYGPVCDNILSVVNSVGQKEDMELLNFYSSTKPAGFNTPVLSNVSTGEFNISLLLTTPSSRRRRSFIEDLLFTSVESVGLPTDDAYKNCTAGPLGFLKDLACAREYNGLLVLPPIITAEMQALYTSSLVASMAFGGITAAGAIPFATQLQARINHLGITQSLLLKNQEKIAASFNKAIGHMQEGFRSTSLALQQIQDVVSKQSAILTETMASLNKNFGAISSVIQEIYQQFDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDVVTLTSCQANYVIVNKTVITTFVDNDDFDFNDELSKWWNDTKHELPDFDKFNYTVPILDIDSEIDRIQGVIQGLNDSLIDLEKLSILKTYIK

SEQ ID NO: 78 - protein, 67 aa, Infectious Bronchitis Virus; identical to SEQ ID NO: 6

SEQ ID NO: 79 - protein, 20 aa, Infectoius Bronchitis Virus
VWLAIAFATIIFILILGWVF

SEQ ID NO: 80 - protein, 47 aa, Infectious Bronchitis Virus; identical to SEQ ID NO: 16

SEQ ID NO: 81 - protein, 1169 aa, Infectious Bronchitis Virus
MLVKSLFLVTILFALCSANLYDNESFVYYYQSAFRPGHGWHLHGGAYAVVNVSSENNNAGTAPSCTAGAIGYSKNLSAASVAMTAPLSGMSWSANSFCTAHCNFTSYIVFVTHCYKSGSNSCPLTGLIPSGYIRIAAMKHGSAMPGHLFYNLTVSVTKYPKFRSLQCVNNHTSVYLNGDLVFTSNYTEDVVAAGVHFKSGGPITYKVMREVKALAYFVNGTAHDVILCDDTPRGLLACQYNTGNFSDGFYPFTNTSIVKDKFIVYRESSVNTTLTLTNFTFSNESGAPPNTGGVDSFILYQTQTAQSGYYNFNFSFLSSFVYRESYYMYGSYHPRCSFRPETLNNGLWFNSLSVSLTYGPIQGGCKQSVFNGKATCCYAYSYGGPRGCKGVYRGELTQHFECGLLVYVTKSDGSRIQTATQPPVLTQNFYNNINLGKCVDYNIYGRIGQGLITNVTDLAVSYNYLSDAGLAILDTSGAIDIFVVQGEYGPNYYKVNPCEDVNQQFVVSGGKLVGILTSRNETGSQLLENQFYIKITNGTRRSRRSVTENVTNCPYVSYGKFCIKPDGSISVIVPKELDQFVAPLLNVTEYVLIPNSFNLTVTDEYIQTRMDKIQINCLQYVCGNSLACRKLFQQYGPVCDNILSVVNSVGQKEDMELLNFYSSTKPARFNTPVFSNLSTGEFNISLLLTPPSSPRRRSFIEDLLFTSVESVGLPTDDAYKKCTAGPLGFLKDLACAREYNGLLVLPPIITAEMQTLYTSSLVASMAFGGITAAGAIPFATQLQARINHLGITQSLLLKNQEKIAASFNKAIGHMQEGFRSTSLALQQIQDVVNKQSAILTETMAALNKNFGAISSVIQDIYQQLDSIQADAQVDRLITGRLSSLSVLASAKQSEYIRVSQQRELATQKINECVKSQSIRYSFCGNGRHVLTIPQNAPNGIVFIHFTYTPESFINVTAIVGFCVSPANASQYAIVPANGRGIFIQVNGSYYITARDMYMPRDITAGDIVTLTSCQANYVSVNKTVITTFVDNDDFDFDDELSKWWNDTKHELPDFDKFNYTVPILDIDSEIDRIQGVIQGLNDSLIDLETLSILKTYIKWPWYVWLAIAFATIIFILILGWLFFMTGCCGCCCGCFGIIPLMSKCGKKSSYYTTFDNDVVTEQYRPKKSV

SEQ ID NO: 82 - protein, 17 aa, Infectious Bronchitis Virus
MLVKSLFLVTILFALCS

SEQ ID NO: 83 - protein, 522 aa, Infectious Bronchitis Virus
ANLYDNESFVYYYQSAFRPGHGWHLHGGAYAVVNVSSENNNAGTAPSCTAGAIGYSKNLSAASVAMTAPLSGMSWSANSFCTAHCNFTSYIVFVTHCYKSGSNSCPLTGLIPSGYIRIAAMKHGSAMPGHLFYNLTVSVTKYPKFRSLQCVNNHTSVYLNGDLVFTSNYTEDVVAAGVHFKSGGPITYKVMREVKALAYFVNGTAHDVILCDDTPRGLLACQYNTGNFSDGFYPFTNTSIVKDKFIVYRESSVNTTLTLTNFTFSNESGAPPNTGGVDSFILYQTQTAQSGYYNFNFSFLSSFVYRESYYMYGSYHPRCSFRPETLNNGLWFNSLSVSLTYGPIQGGCKQSVFNGKATCCYAYSYGGPRGCKGVYRGELTQHFECGLLVYVTKSDGSRIQTATQPPVLTQNFYNNINLGKCVDYNIYGRIGQGLITNVTDLAVSYNYLSDAGLAILDTSGAIDIFVVQGEYGPNYYKVNPCEDVNQQFVVSGGKLVGILTSRNETGSQLLENQFYIKITNGT

SEQ ID NO: 84 - protein, 5 aa, Infectious Bronchitis Virus
RRSRR

SEQ ID NO: 85 - protein, 554 aa, Infectious Bronchitis Virus
SVTENVTNCPYVSYGKFCIKPDGSISVIVPKELDQFVAPLLNVTEYVLIPNSFNLTVTDEYIQTRMDKIQINCLQYVCGNSLACRKLFQQYGPVCDNILSVVNSVGQKEDMELLNFYSSTKPARFNTPVFSNLSTGEFNISLLLTPPSSPRRRSFIEDLLFTSVESVGLPTDDAYKKCTAGPLGFLKDLACAREYNGLLVLPPIITAEMQTLYTSSLVASMAFGGITAAGAIPFATQLQARINHLGITQSLLLKNQEKIAASFNKAIGHMQEGFRSTSLALQQIQDVVNKQSAILTETMAALNKNFGAISSVIQDIYQQLDSIQADAQVDRLITGRLSSLSVLASAKQSEYIRVSQQRELATQKINECVKSQSIRYSFCGNGRHVLTIPQNAPNGIVFIHFTYTPESFINVTAIVGFCVSPANASQYAIVPANGRGIFIQVNGSYYITARDMYMPRDITAGDIVTLTSCQANYVSVNKTVITTFVDNDDFDFDDELSKWWNDTKHELPDFDKFNYTVPILDIDSEIDRIQGVIQGLNDSLIDLETLSILKTYIK

SEQ ID NO: 86 - protein, 67 aa, Infectious Bronchitis Virus
VWLAIAFATIIFILILGWLFFMTGCCGCCCGCFGIIPLMSKCGKKSSYYTTFDNDVVTEQYRPKKSV

SEQ ID NO: 87 - protein, 20 aa, Infectious Bronchitis Virus
VWLAIAFATIIFILILGWLF

SEQ ID NO: 88 - protein, 47 aa, Infectious Bronchitis Virus; identical to SEQ ID NO: 24

SEQ ID NO: 89 - protein, 1168 aa, Infectious Bronchitis Virus
MLVKSLFLVTILFALCSANLYDNESFVYYYQSAFRPGHGWHLHGGAYAVVNVSSENNNAGTAPSCTAGAIGYSKNLSAASVAMTAPLSGMSWSANSFCTAHCNFTSYIVFVTHCYKSGSNSCPLTGLIPSGYIRIAAMKHGSAMPGHLFYNLTVSVTKYPKFRSLQCVNNYTSVYLNGDLVFTSNYTEDVVAAGVHFKSGGPITYKVMREVKALAYFVNGTAHDVILCDDTPRGLLACQYNTGNFSDGFYPFTNTSIVKDKFIVYRESSVNTTLTLTNFTFSNESGAPPNTGGVDSFILYQTQTAQSGYYNFNFSFLSSFVYRESNYMYGSYHPRCSFRPETLNGLWFNSLSVSLTYGPIQGGCKQSVFNGKATCCYAYSYGGPRGCKGVYRGELTQHFECGLLVYVTKSDGSRIQTATQPPVLTQNFYNNINLGKCVDYNIYGRIGQGLITNVTDLAVSYNYLSDAGLAILDTSGAIDIFVVQGEYGPNYYKVNPCEDVNQQFVVSGGKLVGILTSRNETGSQLLENQFYIKITNGTRRSRRSVTENVTNCPYVSYGKFCIKPDGSISVIVPKELDQFVAPLLNVTEYVLIPNSFNLTVTDEYIQTRMDKIQINCLQYVCGNSLACRKLFQQYGPVCDNILSVVNSVGQKEDMELLNFYSSTKPARFNTPVFSNLSTGEFNISLLLTSPSSPRRRSFIEDLLFTSVESVGLPTDDAYKKCTAGPLGFFKDLACAREYNGLLVLPPIITAEMQTLYTSSLVASMAFGGITAAGAIPFATQLQARINHLGITQSLLLKNQEKIAASFNKAIGHMQEGFRSTSLALQQIQDVVNKQSAILTETMAALNKNFGAISSVIQDIYQQLDSIQADAQVDRLITGRLSSLSVLASAKQSEYIRVSQQRELATQKINECVKSQSIRYSFCGNGRHVLTIPQNAPNGIVFIHFTYTPESFINVTAIVGFCVSPANASQYAIVPANGRGIFIQVNGSYYITARDMYMPRDITAGDIVTLTSCQANYVSVNKTVITTFVDNDDFDFDDELSKWWNDTKHELPDFDKFNYTVPILDIDSEIDRIQGVIQGLNDSLIDLETLSILKTYIKWPWYVWLAIAFATIIFILILGWLFFMTGCCGCCCGCFGIIPLMSKCGKKSSYYTTFDNDVVTEQYRPKKSV

SEQ ID NO: 90 - protein, 17 aa, Infectious Bronchitis Virus
MLVKSLFLVTILFALCS

SEQ ID NO: 91 - protein, 521 aa, Infectious Bronchitis Virus
ANLYDNESFVYYYQSAFRPGHGWHLHGGAYAVVNVSSENNNAGTAPSCTAGAIGYSKNLSAASVAMTAPLSGMSWSANSFCTAHCNFTSYIVFVTHCYKSGSNSCPLTGLIPSGYIRIAAMKHGSAMPGHLFYNLTVSVTKYPKFRSLQCVNNYTSVYLNGDLVFTSNYTEDVVAAGVHFKSGGPITYKVMREVKALAYFVNGTAHDVILCDDTPRGLLACQYNTGNFSDGFYPFTNTSIVKDKFIVYRESSVNTTLTLTNFTFSNESGAPPNTGGVDSFILYQTQTAQSGYYNFNFSFLSSFVYRESNYMYGSYHPRCSFRPETLNGLWFNSLSVSLTYGPIQGGCKQSVFNGKATCCYAYSYGGPRGCKGVYRGELTQHFECGLLVYVTKSDGSRIQTATQPPVLTQNFYNNINLGKCVDYNIYGRIGQGLITNVTDLAVSYNYLSDAGLAILDTSGAIDIFVVQGEYGPNYYKVNPCEDVNQQFVVSGGKLVGILTSRNETGSQLLENQFYIKITNGT

SEQ ID NO: 92 - protein, 5 aa, Infectious Bronchitis Virus
RRSRR

SEQ ID NO: 93 - protein, 554 aa, Infectious Bronchitis Virus
SVTENVTNCPYVSYGKFCIKPDGSISVIVPKELDQFVAPLLNVTEYVLIPNSFNLTVTDEYIQTRMDKIQINCLQYVCGNSLACRKLFQQYGPVCDNILSVVNSVGQKEDMELLNFYSSTKPARFNTPVFSNLSTGEFNISLLLTSPSSPRRRSFIEDLLFTSVESVGLPTDDAYKKCTAGPLGFFKDLACAREYNGLLVLPPIITAEMQTLYTSSLVASMAFGGITAAGAIPFATQLQARINHLGITQSLLLKNQEKIAASFNKAIGHMQEGFRSTSLALQQIQDVVNKQSAILTETMAALNKNFGAISSVIQDIYQQLDSIQADAQVDRLITGRLSSLSVLASAKQSEYIRVSQQRELATQKINECVKSQSIRYSFCGNGRHVLTIPQNAPNGIVFIHFTYTPESFINVTAIVGFCVSPANASQYAIVPANGRGIFIQVNGSYYITARDMYMPRDITAGDIVTLTSCQANYVSVNKTVITTFVDNDDFDFDDELSKWWNDTKHELPDFDKFNYTVPILDIDSEIDRIQGVIQGLNDSLIDLETLSILKTYIK

SEQ ID NO: 94 - protein, 67 aa, Infectious Bronchitis Virus; identical to SEQ ID NO: 86

SEQ ID NO: 95 - protein, 20 aa, Infectious Bronchitis Virus
VWLAIAFATIIFILILGWLF

SEQ ID NO: 96 - protein, 47 aa, Infectious Bronchitis Virus; identical to SEQ ID NO: 24

SEQ ID NO: 97 - protein, 1152 aa, Artificial
  Recombinant Spike Ectodomain of Infectious Bronchitis Virus
MPMGSLQPLATLYLLGMLVASVLANLYDNESFVYYYQSAFRPGHGWHLHGGAYAVVNVSSENNNAGTAPSCTAGAIGYSKNLSAASVAMTAPLSGMSWSANSFCTAHCNFTSYIVFVTHCYKSGSNSCPLTGLIPSGYIRIAAMKHGSAMPGHLFYNLTVSVTKYPKFRSLQCVNNYTSVYLNGDLVFTSNYTEDVVAAGVHFKSGGPITYKVMREVKALAYFVNGTAHDVILCDDTPRGLLACQYNTGNFSDGFYPFTNTSIVKDKFIVYRESSVNTTLTLTNFTFSNESGAPPNTGGVDSFILYQTQTAQSGYYNFNFSFLSSFVYRESNYMYGSYHPRCSFRPETLNGLWFNSLSVSLTYGPIQGGCKQSVFNGKATCCYAYSYGGPRGCKGVYRGELTQHFECGLLVYVTKSDGSRIQTATQPPVLTQNFYNNINLGKCVDYNIYGRIGQGLITNVTDLAVSYNYLSDAGLAILDTSGAIDIFVVQGEYGPNYYKVNPCEDVNQQFVVSGGKLVGILTSRNETGSQLLENQFYIKITNGTGGGVPSVTENVTNCPYVSYGKFCIKPDGSISVIVPKELDQFVAPLLNVTEYVLIPNSFNLTVTDEYIQTRMDKIQINCLQYVCGNSLACRKLFQQYGPVCDNILSVVNSVGQKEDMELLNFYSSTKPARFNTPVFSNLSTGEFNISLLLTSPSSPRRRSFIEDLLFTSVESVGLPTDDAYKKCTAGPLGFFKDLACAREYNGLLVLPPIITAEMQTLYTSSLVASMAFGGITAAGAIPFATQLQARINHLGITQSLLLKNQEKIAASFNKAIGHMQEGFRSTSLALQQIQDVVNKQSAILTETMAALNKNFGAISSVIQDIYQQLDSIQADAQVDRLITGRLSSLSVLASAKQSEYIRVSQQRELATQKINECVKSQSIRYSFCGNGRHVLTIPQNAPNGIVFIHFTYTPESFINVTAIVGFCVSPANASQYAIVPANGRGIFIQVNGSYYITARDMYMPRDITAGDIVTLTSCQANYVSVNKTVITTFVDNDDFDFDDELSKWWNDTKHELPDFDKFNYTVPILDIDSEIDRIQGVIQGLNDSLIDLETLSILKTYIKLIKRMKQIEDKIEEIESKQKKIENEIARIKKIKLVPRGSLEWSHPQFEK

SEQ ID NO: 98 - protein, 23 aa, Homo sapiens
MPMGSLQPLATLYLLGMLVASVL

SEQ ID NO: 99 - protein, 521 aa, Infectious Bronchitis Virus; identical to SEQ ID NO: 91

SEQ ID NO: 100 - protein, 5 aa, Artificial
  Glycine-rich spacer sequence
GGGVP

SEQ ID NO: 101 - protein, 554 aa, Infectious Bronchitis Virus; identical to SEQ ID NO: 93

SEQ ID NO: 102 - protein, 38 aa, Artificial
  Modified trimerization domain of Saccharomyces cerevisiae GCN4
RMKQIEDKIEEIESKQKKIENEIARIKKIKLVPRGSLE

SEQ ID NO: 103 - protein, 8 aa, Streptococcus pyogenes
WSHPQFEK

SEQ ID NO: 104 - protein, 539 aa, Infectious Bronchitis Virus
MSVTPLLLVTLLFALCSAAPYDNNSFVYYYQSAFRPFDGWHLHGGAYEVINTTQEFNNAGSNSECTAGAISWSKHFSAASVAMTAPYNGMSWSAQQFCTAHCNFTYFTVFVTHCYKSGVGMCPLTGMIPKKQIRISAMRSGSGPPDLFYNLTVPVTKYPSFKSLQCVNNQTSVYLNGDLVFSSNETIDVSGAGVHFKAGGPITYKVMREVKALAYFVNGTAHDVILCDGTPRGLLACQYNTGNFTDGFYPFTNSSLVKEKFIVYRENSVNTTLVLHNITFSNVTSAPPAGGDISAIFPTYQMVTAQSGYYNFNFSFLSGFVYKVSDFMYGSYHPKCNFRPENLNNDLWFNSLSISLAYGPLQGGCKQSVFNGRATCCYAYSYNGPHACKGVYRGQLQQYFECGLLVYITKSDGSRIQTATKAPVVTTNFYNNITLDRCVDYNIYGRVGQGFITNVTDSTADYNYLADGGLAILDTSGAIDIFVVQGEYGLNFYKVNPCEDVNQQFVVSGGKLVGVLTSRNETGSQFLENQFYIKLTNET

SEQ ID NO: 105 - protein, 17 aa, Infectious Bronchitis Virus
MSVTPLLLVTLLFALCS

SEQ ID NO: 106 - protein, 522 aa, Infectious Bronchitis Virus
AAPYDNNSFVYYYQSAFRPFDGWHLHGGAYEVINTTQEFNNAGSNSECTAGAISWSKHFSAASVAMTAPYNGMSWSAQQFCTAHCNFTYFTVFVTHCYKSGVGMCPLTGMIPKKQIRISAMRSGSGPPDLFYNLTVPVTKYPSFKSLQCVNNQTSVYLNGDLVFSSNETIDVSGAGVHFKAGGPITYKVMREVKALAYFVNGTAHDVILCDGTPRGLLACQYNTGNFTDGFYPFTNSSLVKEKFIVYRENSVNTTLVLHNITFSNVTSAPPAGGDISAIFPTYQMVTAQSGYYNFNFSFLSGFVYKVSDFMYGSYHPKCNFRPENLNNDLWFNSLSISLAYGPLQGGCKQSVFNGRATCCYAYSYNGPHACKGVYRGQLQQYFECGLLVYITKSDGSRIQTATKAPVVTTNFYNNITLDRCVDYNIYGRVGQGFITNVTDSTADYNYLADGGLAILDTSGAIDIFVVQGEYGLNFYKVNPCEDVNQQFVVSGGKLVGVLTSRNETGSQFLENQFYIKLTNET

SEQ ID NO: 107 - protein, 554 aa, Infectious Bronchitis Virus
SVSENVTSCPYVSYGNYCIKPDGSVSLIVPQELEQFVAPLLNVTEHVLLPDSFNLTVTDEYIQTRMDKVQINCLQYVCGNSLECRKLFQQYGPVCDNILSVVNSVGQKEDMELLSFYSSTKPTGYNTPIFNNLTTGAFNVSLLLPQLDSPRGRSFLEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGHMQEGFRSTSLALQQIQDVVNKQSAILTETMQSLNKNFGAISSVIQEIYQQLDTIQANAQVDRLITGRLSSLSVLASAKQAEYIRVSQQRELATQKINECVKSQSIRYSFCGNGRHVLTIPQNAPNGIVFIHFTYTPESFVNVTAIVGFCVNPANASQYAIVPANGRGIFIQVNGSYYITARDMYMPRAITAGDIVMLTSCQANYVSVNKTVITTFVEDDDFDFNDELSKWWNETKHEFPDFDQFNYTIPVLNITYDIDKIEEVIKGLNDSLIDLETLSILKTYIK

SEQ ID NO: 108 - protein, 539 aa, Infectious Bronchitis Virus
MSVLLPLLVTLLCALCSAVLYDINSYVYYYQSAFRPSNGWHLYGGAYAVVNVSNENNNAGSASTCTAGAIGYSKNFSAASIAMTAPPSGMAWSTAAFCTAHCNFTNIVVFVTHCYKSGSGSCPLTGFIQSGYIRISAMKKECSGPSCLFYNLTESVSKYPTFRSLQCVNNYTSVYLNGDLVFTSNYTQDVVAAGVHFKSGGPITYKVMREVKALAYFVNGTAQDVILCDDTPRGLLACQYNTGNFSDGFYPFTNTSIVKDKFIVYRESSVNTTLTLTNFTFSNESGAPPNTGGVNSFILYQTQTAQSGYYNFNFSFLSGFVYEESNYMYGSYHPLCSFRPENINNGLWFNSLSVSITYGPLQGGCKQSFFQGRATCCYAYSYNGPRACKGVYSGELTQSFECGLLVYITKSDGSRIQTATKAPVVTTNFYNNITLDKCVEYNIYGRVGQGFITNVTDSAFGYNYLQDGGLAILDTSGAIDIFVVKGVYGLNYYKVNPCEDVNQQFVVSGGTLVGVLTSRNETGSQFLENQFYIKLTNGT

SEQ ID NO: 109 - protein, 17 aa, Infectious Bronchitis Virus
MSVLLPLLVTLLCALCS

SEQ ID NO: 110 - protein, 522 aa, Infectious Bronchitis Virus
AVLYDINSYVYYYQSAFRPSNGWHLYGGAYAVVNVSNENNNAGSASTCTAGAIGYSKNFSAASIAMTAPPSGMAWSTAAFCTAHCNFTNIVVFVTHCYKSGSGSCPLTGFIQSGYIRISAMKKECSGPSCLFYNLTESVSKYPTFRSLQCVNNYTSVYLNGDLVFTSNYTQDVVAAGVHFKSGGPITYKVMREVKALAYFVNGTAQDVILCDDTPRGLLACQYNTGNFSDGFYPFTNTSIVKDKFIVYRESSVNTTLTLTNFTFSNESGAPPNTGGVNSFILYQTQTAQSGYYNFNFSFLSGFVYEESNYMYGSYHPLCSFRPENINNGLWFNSLSVSITYGPLQGGCKQSFFQGRATCCYAYSYNGPRACKGVYSGELTQSFECGLLVYITKSDGSRIQTATKAPVVTTNFYNNITLDKCVEYNIYGRVGQGFITNVTDSAFGYNYLQDGGLAILDTSGAIDIFVVKGVYGLNYYKVNPCEDVNQQFVVSGGTLVGVLTSRNETGSQFLENQFYIKLTNGT

SEQ ID NO: 111 - protein, 554 aa, Infectious Bronchitis Virus
SVNENVTSCPYVSYGKFCIKPDGSTSVIVPKELEQFVTPLLNATEYVPIPDSFNLTVTDEYIQTRMDKVQINCLQYVCGNSFECRNLFQQYGPVCDNILSIVNSVSQKEDMELLTFYSSTKPFGFNTPILSNLSTGDFNISLLLTPPSSTTGRSFIEDLLFTSVESVGLPTDDAYKKCTAGPLGFLKDLACAREYNGLLVLPPIITAEMQTMYTSSLVASMALGGITAAGAIPFATQLQARINHLGITQAVLLKNQEKIAASFNKAIGQMQEGFRSTSLALQQIQDVVNKQSAILTETMASLNKNFGAISSVIQDIYQQLDVIQADAQVDRLITGRLSSLSVLASAKQSEHIIASQQRELATQKINECVKSQSTRYSFCGNGRHVLTIPQNAPNGIVFIHFTYTPESFVNVTAIVGFCVKPANASQYAIVPANGRGIFIQFNGSYYITARDMYMPRNITAGDIVTLTSCQSNYVSVNKTVITTFVDNDDFDFDDELSKWWNDTKHELPDFDEFNYTAPILDIDSEIDRIQGVIQGLNDSLIDLETLSILKTYIK

SEQ ID NO: 112 - protein, 534 aa, Infectious Bronchitis Virus
MLGKSLFLVTILCALCSANLFDPANYVYYYQSAFRPSNGWHLQGGAYAVVNSSNYANNAGSASECTVGVIKDVYNQSAASIAMTAPLQGMAWSKSQFCSAHCDFSEITVFVTHCYSSGSGSCPITGMIARGHIRISAMKNGSLFYNLTVSVSKYPNFKSFQCVNNFTSVYLNGDLVFTSNKTTDVTSAGVYFKAGGPVNYSIMKEFKVLAYFVNGTAQDVILCDNSPKGLLACQYNTGNFSDGFYPFTNSTLVREKFIVYRESSVNTTLALTNFTFTNVSNAQPNSGGVHTFHLYQTQTAQSGYYNFNLSFLSQFVYKASDYMYGSYHPICAFRPETINSGLWFNSLSVSLTYGPLQGGYKQSVFSGKATCCYAYSYNGPRACKGVYSGELSRDFECGLLVYVTKSDGSRIQTRTEPLVLTQHNYNNITLDKCVAYNIYGRVGQGFITNVTDSVANFSYLADGGLAILDTSGAIDVFVVQGSYGLNYYKVNPCEDVNQQFVVSGGNIVGILTSRNETGSEQVENQFYVKLTNSS

SEQ ID NO: 113 - protein, 17 aa, Infectious Bronchitis Virus
MLGKSLFLVTILCALCS

SEQ ID NO: 114 - protein, 517 aa, Infectious Bronchitis Virus
ANLFDPANYVYYYQSAFRPSNGWHLQGGAYAVVNSSNYANNAGSASECTVGVIKDVYNQSAASIAMTAPLQGMAWSKSQFCSAHCDFSEITVFVTHCYSSGSGSCPITGMIARGHIRISAMKNGSLFYNLTVSVSKYPNFKSFQCVNNFTSVYLNGDLVFTSNKTTDVTSAGVYFKAGGPVNYSIMKEFKVLAYFVNGTAQDVILCDNSPKGLLACQYNTGNFSDGFYPFTNSTLVREKFIVYRESSVNTTLALTNFTFTNVSNAQPNSGGVHTFHLYQTQTAQSGYYNFNLSFLSQFVYKASDYMYGSYHPICAFRPETINSGLWFNSLSVSLTYGPLQGGYKQSVFSGKATCCYAYSYNGPRACKGVYSGELSRDFECGLLVYVTKSDGSRIQTRTEPLVLTQHNYNNITLDKCVAYNIYGRVGQGFITNVTDSVANFSYLADGGLAILDTSGAIDVFVVQGSYGLNYYKVNPCEDVNQQFVVSGGNIVGILTSRNETGSEQVENQFYVKLTNSS

SEQ ID NO: 115 - protein, 554 aa, Infectious Bronchitis Virus
SIGQNVTSCPYVSYGRFCIEPDGSLKMIVPEELKQFVAPLLNITESVLIPNSFNLTVTDEYIQTRMDKVQINCLQYVCGNSLECRKLFQQYGPVCDNILSVVNSVSQKEDMELLSFYSSTKPKGYDTPVLSNVSTGEFNISLLLTPPSSPSGRSFVEDLLFTSVETVGLPTDAEYKKCTAGPLGTLKDLICAREYNGLLVLPPIITADMQTMYTASLVGAMAFGGITSAAAIPFATQIQARINHLGIAQSLLMKNQEKIAASFNKAIGHMQEGFRSTSLALQQVQDVVNKQSAILTETMNSLNKNFGAISSVIQDIYAQLDAIQADAQVDRLITGRLSSLSVLASAKQSEYIRVSQQRELATQKINECVKSQSNRYGFCGSGRHVLSIPQNAPNGIVFIHFTYTPETFVNVTAIVGFCVNPLNASQYAIVPANGRGIFIQVNGTYYITSRDMYMPRDITAGDIVTLTSCQANYVNVNKTVITTFVEDDDFNFDDELSKWWNDTKHGLPDFDDFNYTVPILNISGEIDNIQGVIQGLNDSLINLEELSIIKTYIK

SEQ ID NO: 116 - protein, 529 aa, Infectious Bronchitis Virus
MLVKSLFIVTLLLALCEGGLVGVNYTYYYQSGYRPPNGWHMQGGAYKVVNKTTISYTNQGCTIGVIRGGVTINQSAIAFTSATGSSWSKQGVCTVYCNYTSFYVFVTHCGGTGHNCIVNTKQLGVLVFGVKNYNDQFIYNRTLGAAGPYANFKAWQCLSNYTSVFLNGNLLYTSNYTEDVKAAGVYAKQVNGLERRVMRDTPVMAYFVNGTVQDVILCDDSPKGRLACQYNTGNFSDGLYPVYEEPVASNFTFVPLDTSSTSYGVLHNFTFNNVTGVAPNQNHIARFNISTISEGYVNFKFNFLNSFTYVESDFDRGSYYGKPGSRCNFRLESINRGLSFNSLTVSIGYGPISGGCKQSVWKNEATCCFAYKYNGGSRNCKGLYTFDSDVNYECVLLVFISKTDGSRIRTATSPPVYSNNNVNINLGLCVDYNVYGITGRGLITNITESVHPGYLDHGGLVLLDATGSIDTFVLHSDNLTSYYKVNPCSDINEQYVVSGGNLVGKLTSNNQTVAQQLGDMFYVKFSTSG

SEQ ID NO: 117 - protein, 17 aa, Infectious Bronchitis Virus
MLVKSLFIVTLLLALCE

SEQ ID NO: 118 - protein, 512 aa, Infectious Bronchitis Virus
GGLVGVNYTYYYQSGYRPPNGWHMQGGAYKVVNKTTISYTNQGCTIGVIRGGVTINQSAIAFTSATGSSWSKQGVCTVYCNYTSFYVFVTHCGGTGHNCIVNTKQLGVLVFGVKNYNDQFIYNRTLGAAGPYANFKAWQCLSNYTSVFLNGNLLYTSNYTEDVKAAGVYAKQVNGLERRVMRDTPVMAYFVNGTVQDVILCDDSPKGRLACQYNTGNFSDGLYPVYEEPVASNFTFVPLDTSSTSYGVLHNFTFNNVTGVAPNQNHIARFNISTISEGYVNFKFNFLNSFTYVESDFDRGSYYGKPGSRCNFRLESINRGLSFNSLTVSIGYGPISGGCKQSVWKNEATCCFAYKYNGGSRNCKGLYTFDSDVNYECVLLVFISKTDGSRIRTATSPPVYSNNNVNINLGLCVDYNVYGITGRGLITNITESVHPGYLDHGGLVLLDATGSIDTFVLHSDNLTSYYKVNPCSDINEQYVVSGGNLVGKLTSNNQTVAQQLGDMFYVKFSTSG

SEQ ID NO: 119 - protein, 554 aa, Infectious Bronchitis Virus
ATSENVISCPYVTYGKFCIKPDGGISNIVPEEVKDYTSLLLNRTDYVLIPNSFNLTVTDEFIQTQFQKIQINCIQYVCGSSIQCKQLFQQYGSVCDSILSIVNGIALQDNAEMLHFYSSTKPRGFDTNSFVNFTAGEFNISLVLPKNGQPTGRSFIEDLLFDKVESLGLPGDSAYQKCTSGPLGFVKDLVCAQNYNGLLVLPPIITAEMQAMYTSSLVASMAFGGITSAGAIPFATQIQARINHLGITQTVLQKNQEKIAASFNKAMKHMQDGFSATSLALQQVQDVVNEQGAILQQTMHSLNKNFGAISHVIQDIYKQLDALEANAQVDRIITGRLSSLSVLASAKQLEYTKVSQQRELAKEKINECVKSQSNRHGFCGEGMHIMSIPQNAPNGIVFLHFTYTPETYANVTAVVGFCVKPGNGTEYGLVPVVGRGIFIEVNGTYYITGRDMYSPRAITAGDVVKLTSCQANYQSINRTVITTFVDEDDFDFDHELSKWWNETSRDFPNLDEFNYTIPVLNISNEIDKIQEVIQGLNDSIIDLETLSILKTYIK

SEQ ID NO: 120 - protein, 1115 aa, Artificial Sequence
  Synthetic
NLYDNESFVYYYQSAFRPGHGWHLHGGAYAVVNVSSENNNAGTAPSCTAGAIGYSKNLSAASVAMTAPLSGMSWSANSFCTAHCNFTSYIVFVTHCYKSGSNSCPLTGLIPSGYIRIAAMKHGSAMPGHLFYNLTVSVTKYPKFRSLQCVNNYTSVYLNGDLVFTSNYTEDVVAAGVHFKSGGPITYKVMREVKALAYFVNGTAHDVILCDDTPRGLLACQYNTGNFSDGFYPFTNTSIVKDKFIVYRESSVNTTLTLTNFTFSNESGAPPNTGGVDSFILYQTQTAQSGYYNFNFSFLSSFVYRESNYMYGSYHPRCSFRPETLNGLWFNSLSVSLTYGPIQGGCKQSVFNGKATCCYAYSYGGPRGCKGVYRGELTQHFECGLLVYVTKSDGSRIQTATQPPVLTQNFYNNINLGKCVDYNIYGRIGQGLITNVTDLAVSYNYLSDAGLAILDTSGAIDIFVVQGEYGPNYYKVNPCEDVNQQFVVSGGKLVGILTSRNETGSQLLENQFYIKITNGTGGGVPSVTENVTNCPYVSYGKFCIKPDGSISVIVPKELDQFVAPLLNVTEYVLIPNSFNLTVTDEYIQTRMDKIQINCLQYVCGNSLACRKLFQQYGPVCDNILSVVNSVGQKEDMELLNFYSSTKPARFNTPVFSNLSTGEFNISLLLTSPSSPRRRSFIEDLLFTSVESVGLPTDDAYKKCTAGPLGFLKDLACAREYNGLLVLPPIITAEMQTLYTSSLVASMAFGGITAAGAIPFATQLQARINHLGITQSLLLKNQEKIAASFNKAIGHMQEGFRSTSLALQQIQDVVNKQSAILTETMAALNKNFGAISSVIQDIYQQLDSIQADAQVDRLITGRLSSLSVLASAKQSEYIRVSQQRELATQKINECVKSQSIRYSFCGNGRHVLTIPQNAPNGIVFIHFTYTPESFINVTAIVGFCVSPANASQYAIVPANGRGIFIQVNGSYYITARDMYMPRDITAGDIVTLTSCQANYVSVNKTVITTFVDNDDFDFDDELSKWWNDTKHELPDFDKFNYTVPILDIDSEIDRIQGVIQGLNDSLIDLETLSILKTYIKRMKQIEDKIEEIESKQKKIENEIARIKKLVPRGSLE

SEQ ID NO: 121 - protein, 126 aa, Gallus gallus
PTTTYSCCYKVYTILEEITSHLESTAATAGLSSVPMDIRDKTCLRNNLKTFIESLKTNGTEEESGIVFQLNRVHECERLFSNITPTPQVPDKECRTAQVSREKFKEALKTFFIYLSDVLPEEKDCI

SEQ ID NO: 122 - protein, 36 aa, Artificial Sequence
  Synthetic- multimerization domain
RMKQIEDKIEEIESKQKKIENEIARIKKLVPRGSLE

SEQ ID NO: 123 - protein, 33 aa, Artificial Sequence
  Synthetic- multimerization domain
RMKQIEDKIEEILSKIYHIENEIARIKKLIGER

SEQ ID NO: 124 - DNA, 22952 bp, Artificial Sequence
  Synthetic-pLS-GMCSF-ARKSe
ggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgatccggctgctaacaaagcccgaaaggaagctgagttggctgctgccaccgctgagcaataactagcataaccccttggggcctctaaacgggtcttgaggggttttttgctgaaaggaggaactatatccggatggccgccaccggtgggccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacgattcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcggaagcggccgctaatacgactcactatagggaccaaacagagaatccgtgagtcgcgataaaaggcgaaagagcaattgaagtcacacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggataggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgagcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaaaacaaacaacgtctgtagcgaccctttgcaggcagcggaaccccccacctggcgacaggtgcctctgcggccaaaagccacgtgtataaaatacacctgcaaaggcggcacaaccccagtgccacgttgtgagttggatagttgtggaaagagtcaaatggctctcctcaagcgtattcaacaaggggctgaaggatgcccaaaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgaccaccacatattcatgctgctacaaagtgtataccattctggaggaaatcacatcccacctggagtctactgccgcaactgccggcctgagctccgtgccaatggatatcagagacaagacatgcctgaggaacaatctgaagactttcattgaaagcctgaaaactaacggcaccgaggaagagtccggaatcgtgttccagctgaaccgcgtgcacgaatgcgagcggctgtttagcaatattacccccacacctcaggtgcccgataaagagtgcagaaccgcccaggtgtcccgcgaaaagtttaaagaggctctgaaaaccttctttatctacctgagcgatgtgctgcctgaggaaaaggattgtatctaatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaaggcaaccccgatcgacaggacagatctgacaaacaaccatccacacccgggcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggtcgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctccaagcggcaatcctctctcgcttcttcagccccactgaatgatcgcgtaaccgtaattaatctagctacattaaggattaagaaaaaatacgggtagaattggagtgccccaattgtgagtttagttgatagttgtagccaccatgcctatgggttcactgcagcctctggctactctgtatctgctgggaatgctggtggcctccgtgctggctaatctgtatgacaacgagagctttgtgtactactaccagtccgctttcagacctggacacggatggcacctgcacggcggagcctacgctgtggtgaatgtgagctccgaaaacaataatgcagggacagctcctagctgcactgcaggagcaattggatactccaaaaacctgagcgccgcttccgtggctatgacagcacctctgagcgggatgagctggtccgctaattccttctgcaccgcacactgcaactttacaagctacatcgtgttcgtgactcactgctacaaaagcggatccaatagctgccctctgaccgggctgattccatccggctacatcaggattgcagccatgaagcacggaagcgccatgccagggcacctgttttacaacctgactgtgagcgtgaccaagtaccccaaattccgctccctgcagtgcgtgaataactacaccagcgtgtacctgaatggcgatctggtgtttacatccaactacactgaggacgtggtggctgcaggagtgcacttcaagagcgggggccccatcacatacaaagtgatgagagaagtgaaggctctggcatacttcgtgaacggaacagctcacgatgtgattctgtgcgatgacactcctcgcgggctgctggcatgccagtacaatacaggcaacttttccgatggattttacccattcacaaatactagcatcgtgaaggacaaattcattgtgtaccgggagagctccgtgaacaccacactgaccctgacaaatttcacttttagcaacgaatccggagcccctcccaataccggaggggtggacagctttatcctgtaccagactcagaccgctcagagcggctactacaatttcaacttttccttcctgagctccttcgtgtacagagagtccaactacatgtacggaagctaccacccccggtgctcctttagacctgaaaccctgaatgggctgtggttcaactccctgagcgtgtccctgacatacggcccaatccagggcggatgcaaacagtccgtgttcaacggaaaggcaacctgctgctacgcatactcctacggcggaccaagaggatgcaaaggagtgtacagaggggagctgacccagcactttgaatgcggcctgctggtgtacgtgacaaagagcgatggctcccggatccagacagctactcagccacccgtgctgacacagaatttctacaataacattaacctgggaaagtgcgtggactacaacatctacgggagaattggacaggggctgatcactaatgtgaccgatctggcagtgtcctacaactacctgagcgacgccggactggctattctggataccagcggggccatcgacatttttgtggtgcagggggagtacggccctaattactacaaagtgaacccatgcgaagatgtgaatcagcagttcgtggtgagcggagggaagctggtgggcatcctgacaagcagaaacgagactggatcccagctgctggaaaaccagttctacatcaagatcactaatggaactggcggaggggtgccatccgtgaccgagaatgtgacaaactgcccttacgtgagctacggcaaattctgcatcaagcccgatggaagcatctccgtgattgtgcctaaggagctggaccagtttgtggccccactgctgaacgtgactgaatacgtgctgatccctaattccttcaacctgaccgtgacagatgagtacattcagacccggatggacaaaatccagattaattgcctgcagtacgtgtgcgggaacagcctggcatgcagaaagctgttccagcagtacggcccagtgtgcgataatatcctgagcgtggtgaactccgtgggccagaaagaggacatggaactgctgaatttttacagctccacaaagccagccaggtttaatactcccgtgttcagcaacctgtccaccggagagttcaacatctccctgctgctgacaagccccagctcccctagaaggcgcagctttattgaggatctgctgttcactagcgtggaatccgtgggcctgcccaccgatgacgcctacaagaaatgcacagctggacctctggggttctttaaagacctggcctgcgctcgcgagtacaacggactgctggtgctgcctccaatcattaccgccgaaatgcagaccctgtacacaagctccctggtggcaagcatggcctttggcggaatcactgccgctggggctattcctttcgcaacccagctgcaggccaggatcaaccacctgggcattacacagtccctgctgctgaaaaatcaggagaagatcgcagccagctttaacaaggctattgggcacatgcaggaaggcttccgcagcacttccctggcactgcagcagatccaggacgtggtgaacaaacagagcgccattctgactgagacaatggctgcactgaataagaactttggggctatcagctccgtgatccaggatatttaccagcagctggacagcatccaggcagatgcccaggtggacaggctgattactggaagactgagctccctgagcgtgctggcttccgcaaagcagagcgaatacatccgggtgtcccagcagagagagctggcaacacagaaaatcaatgaatgcgtgaagagccagtccattaggtacagcttctgcggcaacggacgccacgtgctgactattccacagaatgctcccaacggcatcgtgtttattcacttcacatacactcccgagagctttatcaatgtgaccgcaattgtgggattctgcgtgtccccagccaacgctagccagtacgccatcgtgcccgctaatgggcggggcatctttattcaggtgaacggaagctactacattacagcaagggatatgtacatgcctcgcgatatcactgccggggacattgtgaccctgacaagctgccaggctaattacgtgtccgtgaacaaaaccgtgatcactaccttcgtggataacgatgactttgacttcgatgacgagctgtccaaatggtggaatgatacaaagcacgaactgcctgattttgacaagttcaactacactgtgccaatcctggatattgacagcgagatcgaccgcattcagggcgtgatccagggactgaatgatagcctgatcgacctggaaactctgtccattctgaagacctacatcaagctgattaaacggatgaagcagatcgaggacaaaatcgaggaaattgaaagcaagcagaagaaaattgagaacgaaatcgccagaattaaaaagattaagctggtgcctcggggctccctggaatggtcacatcctcagtttgaaaagtgataagtgtgtaactaccgtgtactaagccccactcacccagatcatcatgacacaaaaaactaatcgttacctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatgctgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatccctgctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatggtgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaaaactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaacccgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggtaaataatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgatagaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacgatcaaattgttcgctcatgccatcaatcagttgatgggcctacctcatttcttcgagtggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagatgactctccggagatggtgttgacacagttgcatcaagccagtgataatttcttcaaggaattaatccatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacactgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgcaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgatgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcctgtcattcattcaaggttacatgcagtgggcctggtcaaccatgacggatcacaccaacttgcagatacggattttatcgaaatgtctgcaaaactgttagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatcccatctcctttctgtacccgaggtaagatgtgcaagacacgggaactccttatacttggctgaaggaagcggagccatcatgagtcttcttgaactgcatgtaccacatgaaactatctattacaatacgctcttttcaaatgagatgaaccccccgcaacgacatttcggaccgaccccaactcagtttttgaattcggttgtttataggaatctacaggcggaggtaacatgcaaggatggatttgtccaagagttccgtccattatggagagaaaatacagaggaaagtgacctgacctcagataaagcagtggggtatattacatctgcagtaccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggggatatggagtgttacctggtatttgtcatgggttacctgggcgggcctacatttgtacatgaggtggtgaggatggcaaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagccacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcctaacgaaaatcacatattaataggctccttttttggccaattgtattcttgttgatttaattatattatgttagaaaaaagttgaactctgactccttaggactcgaattcgaactcaaataaatgtctttaaaaaaggttgcgcacaattattcttgagtgtagtctcgtcattcaccaaatctttgtttggtggccggcatggtcccagcctcctcgct